(19) United States
(12) Patent Application Publication
(10) Pub. No.: US 2006/0058227 A1
(43) Pub. Date: Mar. 16, 2006

(54) Hedgehog-related prophylaxis, therapy and diagnosis of gi tract carcinogenesis

(75) Inventors: Gijs Robert Van Den Brink, Amsterdam (NL); Maikel Petrus Peppelenbosch, Utrecht (NL); James Christopher Henry Hardwick, Amsterdam (NL); Sander Jan Hendrik Van Deventer, Haalem (NL)

Correspondence Address: MCKENNA LONG & ALDRIDGE LLP, 1900 K STREET, NW, WASHINGTON, DC, 20006, US

(73) Assignee: Academisch Ziekenhulis bij de Universiteit van Amsterdam, NL-1105 AZ AMSTERDAM-Zuidoost (NL)

(21) Appl. No.: 10/505,230

(22) PCT Filed: Feb. 20, 2003
(86) PCT No.: PCT/NL03/00127

§ 371 Date: Jul. 1, 2005

(30) Foreign Application Priority Data
Feb. 20, 2002 (EP) 02075690.4

(51) Int. Cl. A61K 38/17; C07K 14/47

(52) U.S. Cl. 514/12; 530/350

(57) ABSTRACT

The present invention is based on the key roles played by Hedgehog proteins in the regulation of homeostasis of the adult intestinal epithelium. Ihh is expressed in the adult mammalian colon and provides a lineage-instructive signal and regulates colonic epithelial morphogenesis in a compartmental fashion. Loss of Ihh expression precedes morphological change in colon tumorigenesis, i.e. carcinogenesis, and Ihh was absent in HT-29 colon carcinoma cells. Treatment of cancerous HT-29 cells with exogenous Hedgehog protein restored their differentiation. Ihh thus plays a pivotal role in the maintenance of colonic epithelial homeostasis in the differentiation of the GI tract cells and is essential for the enrolment of these GI tract cells on the Death program thus maintaining homeostasis to avoid or treat carcinogenesis. In addition, in gastric cancer expression of Shh is lost and loss of Shh expression precedes morphological changes in the parietal cells of the stomach. Shh is specifically expressed in fundic glands as well as in gastric heterotopia in the esophagus in Meckel's diverticulum. Shh thus has a unique role as a morphogen in fundic gland homeostasis. The present invention relates to methods in which a source of Hedgehog proteins is used prophylactically or therapeutically to maintain homeostasis of the adult intestinal epithelium. In particular the invention relates methods whereby sources of Hedgehog protein are used to prevent or treat carcinogenesis in adult gastric and colonic tissues. The invention also relates to Hedgehog-based method of diagnosing susceptibility for or the presence of carcinogenesis in the adult GI tract, particularly in gastric and colonic tissues. The invention further provide for compositions to be used in the Hedgehog-based methods of diagnosing, preventing and treating epithelial tumorigenesis in the adult GI tract.

### FIELD OF THE INVENTION

The present invention resides in the fields of recombinant genetics, and medicine and is directed to the use of Indian and Sonic Hedgehog proteins and nucleic acids encoding the Indian and Sonic Hedgehog proteins in the prophylaxis, therapy and diagnosis of GI tract carcinogenesis, e.g. in gastric or colonic cancer.

### BACKGROUND OF THE INVENTION

During organogenesis the cells of the endodermal layer give rise to the liver, pancreas and epithelial cells of the lung and gastrointestinal (GI) tract. The differentiation of these different organs with their respective functional cell types occurs through complex mesenchymal-endodermal interaction. In this interaction, the Hedgehog (hh), Fibroblast Growth Factor (Fgf), Wnt and Transforming Growth Factor (TGF)-β families of secreted proteins play a key role (Hogan, 1999).

Hedgehog was initially identified in a genetic screen for segment polarity genes in Drosophila (Nüsslein-Volhard and Wieschaus, 1980). In vertebrates three hedgehog genes have been identified, Sonic Hedgehog (Shh), Indian Hedgehog (Ihh) and Desert Hedgehog. All three Hedgehog's bind to the same receptor, Patched (Ptc) which controls the activity of a second receptor, Smoothened (Smo) (Kalderon, 2000). Both Shh and Ihh play a role in endodermal/ectodermal-mesodermal interactions in the gut (Bitgood and MacMahon 1995; Roberts et al., 1995; Roberts et al., 1998; Litingung et al., 1998; Sukegawa et al., 2000; Ramalho-Santos et al., 2000; Kim et al., 1998; Hebrok et al., 1998; Apelqvist et al, 1997; Marigo et al. 1995)

Expression of Shh in the gastrointestinal tract has been described during development in many vertebrate systems including mouse (Bitgood et al., 1995), chick (Ronberts et al. 1995), human (Ryan et al., 1998), and frog. In all species examined, Shh is expressed from the earliest time points of gastrointestinal development restricted in its expression to the endoderm. The murine gut has been well examined for Shh mRNA expression throughout development. At a late stage of development, 18.5 days post coitum (d.p.c.), one day prior to birth, Shh mRNA is detected in the glandular epithelium of the stomach, the small intestine and the colon (Apelqvist et al., 50). Thus, Shh is expressed at a late stage of intra-uterine development, whereas at the same time the murine GI tract undergoes major morphological and functional changes during the first three postnatal weeks, including formation of intestinal crypts and maturation of the gastric glands (Gordon and Hermiston, 1994; Karam et al, 1997). No information is available in the art, however, as to what happens to Shh mRNA expression in the adult.

Several studies have addressed the functional role of Shh expression in the developing gut. Studies in chick and mouse using either over expression or inactivation of Shh suggest that during development Shh is a critical endodermal signal in the epithelial/mesodermal signalling involved in specification of differentiation along the anterior-posterior as well as the radial axis of the vertebrate gut (Bitgood and MacMahon 1995; Roberts et al., 1995; Roberts et al., 1998; Litingung et al., 1998; Sukegawa et al., 2000; Ramalho-Santos et al., 2000; Kim et al., 1998; Hebrok et al., 1998; Apelqvist et al, 1997; Marigo et al. 1995). Shh null mice display gastrointestinal malformations including a failure of the trachea and esophagus to separate normally, gut malrotation, small intestinal and anus atresias. The gastric epithelium of Shh null mice shows epithelial hyperplasia and alkaline phosphatase expression, a sign of intestinal differentiation (Apelqvist et al., 1997).

Despite this knowledge of the embryonic and not yet weaned mice, there is a lack of information about Hedgehog expression in the adult and their role in this rapidly regenerating system. Van den Brink et al. (2001) previously showed that Shh is expressed in the fundic gland of the adult human and rodent stomach. Inhibition of Shh led to enhanced epithelial proliferation and diminished protein levels of BMP-4, Islet-1, and Hepatocyte Nuclear Factor 3β, all of which are proteins involved in differentiation and tissue specific gene expression. Although this may indicate some role for Shh in the regulation of fundic gland homeostasis in the adult proximal stomach, there is, however, no insight into the lineage-instructive mechanisms that regulate differentiation of intestinal epithelial precursor cell descendants.

After the establishment of differentiation of the GI tract along all its axes of development, continuous renewal of GI epithelial cells in the adult occurs along a single vertical (or radial) axis. For instance, a common progenitor cell in the crypts of the intestine can give rise to a variety of epithelial cell types with digestive, absorptive, protective and endocrine functions (Stappenbeck et al., 1998; Montgomery et al., 1999; Roberts 2000). The cells differentiate as they move towards the intestinal lumen and undergo a death program thus maintaining homeostasis (Hall et al., 1994). Although loss of this tightly regulated epithelial differentiation is a central aspect of the development of colon cancer, the factors that regulate epithelial differentiation still remain to be identified. Differentiation of the epithelial cells is a cell non-autonomous process that seems to be critically dependent on positional information along the vertical axis of renewal (Sweetser et al., 1988; Hermiston et al., 1996). Several tissue and cell lineage-specific transcription factors have been identified that regulate the expression of cell type specific markers of differentiation (Montgomery et al., 1999). However, the molecular mechanisms that time and direct the induction of these transcription factors at the appropriate position along the vertical axis remained unresolved thus far.

In the adult vertebrate colon, precursor cells at the bottom of the colonic crypt can differentiate in to three cell types: absorptive columnar cells, goblet cells and endocrine cells (Chang et al., 1971). The endocrine cell is present in highest numbers at the base of the crypt whereas most of the goblet cells are located in the mid-crypt. The predominant cell type, the absorptive enterocyte, shows a graded pattern from a relatively undifferentiated phenotype in the crypt to a fully differentiated phenotype at the inter-crypt table (Chang et al., 1971). During embryogenesis cells receive the positional information that determines their developmental fate from their relation to gradients of secreted morphogens (Hogan. 1999).

During development the GI tract is patterned through endodermal-mesenchymal interactions. In this interplay Sonic Hedgehog (Shh) and Indian Hedgehog (Ihh) are critical endodermally derived morphogens (Roberts et al. 1995; Apelqvist et al., 1997; Roberts et al., 1998; Litingung et al., 1998; Ramalho-Santos et al., 2000; Mo et al., 2001; Sukegawa et al. 2000; Zhang et al. 2001). Both have partially overlapping functions and act through the same complex of an Hedgehog binding receptor Patched (Ptc), a signalling receptor Smoothened (Smo) and the Gli family of transcriptional effectors (McMahon 2000). Van den Brink et al. (2001) previously showed that Shh is involved in the regulation of fundic gland homeostasis in the adult proximal stomach. Hedgehog signalling plays an important role in the development of the hindgut and this role is conserved from fly to mice (Hoch and Pancratz 1996; Takashima and Murakami 2001). Ihh mRNA is produced in the colon until at least one day prior to birth in mice (Ramalho-Santos et al., 2000). However, the prior art does not disclose anything on the expression of Ihh mRNA or protein in the adult colon.

Thus, there is still a need for identification of the key molecule(s) that regulate GI tract epithelial homeostasis. As a consequence there is still a need for compositions and methods based on such key molecule(s), that may be used in the prophylaxis, therapy and/or diagnosis of GI tract carcinogenesis, such as e.g. in gastric or colonic cancer.

### DESCRIPTION OF THE INVENTION

### DEFINITIONS

The term “Hedgehog” or “Hedgehog protein” is herein understood to mean a polypeptide with the amino acid sequence that is substantially similar to the amino acid sequences of SEQ ID NO: 1, SEQ ID NO: 2, and SEQ ID NO: 3 (Desert, Indian and Sonic). The term “Hedgehog” is thus interchangeably used for the human Desert, Indian and Sonic Hedgehog proteins (referred to as Dhh, Ihh and Shh, respectively) as well as for their non-human mammalian homologues and includes allelic forms and muteins of these polypeptides comprising one or more amino acid substitutions, deletions and/or insertions. The term “Hedgehog” as used herein thus comprises polypeptides preferably having at least 63% amino acid identity with the amino acid sequence of SEQ ID NO: 1, SEQ ID NO: 2 and SEQ ID NO: 3, preferably having at least 75, 80, 90, 95 or 99% % amino acid identity with the amino acid sequence of SEQ ID NO: 1, SEQ ID NO: 2 and SEQ ID NO: 3.

Included in the term “Hedgehog proteins” are also proteins of the same or similar sequence as a native Hedgehog protein, but lacking amino acid sequences at either or both of its N-terminal and C-terminal ends. Preferably such truncation mutants correspond to the amino terminal half of a “mature” Hedgehog protein. The truncation mutants preferably comprise at least 50-60 amino acid residues, more preferably 90-100 amino acid residues, and most preferably at least 150 amino acid residues of a Hedgehog protein, or variant thereof, while retaining at least one activity of a Hedgehog protein. Such truncated Hedgehog preferably have at least 63% amino acid identity with the amino acid sequence of SEQ ID NO: 1, SEQ ID NO: 2 and SEQ ID NO: 3, preferably having at least 75, 80, 90, 95 or 99% % amino acid identity with the amino acid sequence of SEQ ID NO: 1, SEQ ID NO: 2 and SEQ ID NO: 3.

Any protein, polypeptide or truncated mutant comprised within the term “Hedgehog protein” preferably has at least one biological activity of the native Hedgehog proteins defined by the amino acid sequences of SEQ ID NO: 1, SEQ ID NO: 2 and SEQ ID NO: 3. Preferably, the biological activity of the Hedgehog proteins for use in the present invention at least comprises one or more of: (1) the ability to bind to a Hedgehog binding receptor Patched (Ptc), and preferably activate signalling downstream of Ptc through the receptor Smoothened (Smo) and the Gli family of transcriptional effectors (McMahon, 2000); (2) the ability to maintain homeostasis of; (3) the ability to restore differentiation of; and, (4) the ability to cause gastric and/or colonic epithelial tumour cells to enter the Death program. A suitable assay for the biological activity of a Hedgehog protein is to test its ability to restore differentiation of the HT-29 colonic cancer cell line in vitro, as described in the Examples herein.

The amino acid identity between a polypeptide comprised in the term “Hedgehog” and SEQ ID NO: 1, SEQ ID NO: 2, or SEQ ID NO: 3 may be readily calculated by known methods, including but not limited to those described in (Computational Molecular Biology, Lesk, A. M., ed., Oxford University Press, New York, 1988; Biocomputing: Infomatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). Preferred methods to determine identity are designed to give the largest match between the sequences tested. Methods to determine identity are codified in publicly available computer programs. Preferred computer program methods to determine identity and similarity between two sequences include, but are not limited to, the GCG program package (Devereux, J., et al., Nucleic Acids Research 12 (1):387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity. Preferred parameters for polypeptide sequence comparison include the following: 1) Algorithm: Needleman and Wunsch, J. Mol. Biol. 48:443-453 (1970) Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for peptide comparisons (along with no penalty for end gaps).

Operably Linked

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

Promoter

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is only active in specific types of differentiated cells/tissues.

### DETAILED DESCRIPTION OF THE INVENTION

The present invention is based on the surprising discovery of the key roles played by Hedgehog proteins in the regulation of homeostasis of the adult intestinal epithelium. We have found that Ihh is expressed in the adult human and rodent colon, and that Ihh provides a lineage-instructive signal and regulates colonic epithelial morphogenesis in a compartmental fashion. Loss of Ihh expression precedes morphological change in colon tumorigenesis, i.e. carcinogenesis, and Ihh was absent in HT-29 colon carcinoma cells. Treatment of cancerous HT-29 cells with exogenous Hedgehog protein restored their differentiation. Ihh thus plays a pivotal role in the maintenance of colonic epithelial homeostasis in the differentiation of the GI tract cells and is essential for the enrolment of these GI tract cells on the Death program thus maintaining homeostasis to avoid or treat carcinogenesis.

In addition, in gastric cancer expression of Shh was found to be lost and loss of Shh expression was found to precede morphological changes in the parietal cells of the stomach. Expression of Shh was analysed along the normal human and rodent adult GI tract as well as in intestinal metaplasia of the stomach, gastric and intestinal metaplasia of the esophagus and gastric heterotopia in Meckel's diverticulum. We found that Shh is specifically expressed in fundic glands as well as in gastric heterotopia in the esophagus in Meckel's diverticulum and that Shh has a unique role as a morphogen in fundic gland homeostasis.

Thus, the invention relates to Hedgehog (hh) proteins and their role in maintaining adult intestinal homeostasis. In particular the invention relates to Sonic Hedgehog (Shh) and Indian Hedgehog (Ihh) expression in adult gastric and colonic, tissues respectively, whereby absence or expression of these Hedgehog proteins (or mRNAs) leads to carcinogenesis in these tissues. While it was known in the art before the present invention that Hedgehog is involved in ontogenesis in various types of tissues no understanding was available regarding the role of Hedgehog in adult tissues, in particular no information was available as to the role of Hedgehog in suppressing tumorigenesis in these tissues. We have now found that upregulation of Hedgehog prevents, as well as provides for a treatment of carcinogenesis in the adult gastric and colonic tissues.

The present invention thus provides for methods of, and compositions for use in methods of diagnosis, prevention and therapy of intestinal epithelial tumorigenesis, in particular carcinogenesis of gastric and colonic tissues, using compositions comprising Hedgehog proteins or nucleic acids coding therefor, or compositions for the detection of these Hedgehog molecules.

Thus in a first aspect, the present invention relates to a method of treating an deficiency of a Hedgehog protein in the GI tract, wherein the method comprises providing a source of Hedgehog protein to the GI tract of a subject suffering from the deficiency of a Hedgehog protein in the GI tract. The deficiency of the Hedgehog protein preferably is an acquired deficiency of the Hedgehog protein. The acquired deficiency of the Hedgehog protein in the GI tract may be the result of an acquired somatic mutation resulting in reduced expression of Hedgehog and/or a somatic activating mutation in the Wnt-β-catenin pathway. In the method, the source of Hedgehog protein may be provided to the GI tract of a subject suffering from the deficiency of a Hedgehog protein for the prophylaxis of carcinogenesis in the GI tract. Preferably the source of Hedgehog protein is provided for the prophylaxis of gastric or colonic cancer. Alternatively, the method comprises providing a source of Hedgehog protein to the GI tract of a subject suffering from the acquired deficiency of a Hedgehog protein for the treatment of a GI tract carcinoma. Preferably the source of Hedgehog protein is provided for the treatment of gastric or colonic cancer.

In a preferred embodiment the invention relates to methods of treating a subject having been diagnosed with familial adenomatous polyposis coli (FAP). The method comprises administering to a subject having been diagnosed with FAP source of Hedgehog protein to the GI tract of the subject. The method preferably is a method that prevents or reverses tumorigenesis in the subject having been diagnosed with FAP. Preferably the method is a method that prevents or treats GI tract tumours, in particular, (colonic) adenomatous polyps and invasive adenocarcinomas, small intestinal adenomas and cancers, and desmoid tumors.

In these prophylactic and therapeutic methods, the source of Hedgehog protein is administered in such an amount that functional levels of Hedgehog protein is maintained or restored in the subject's GI tract. The functional level of Hedgehog protein achieves the desired prophylactic or therapeutic effects. Such a functional level preferably is a level that maintains homeostasis of gastric and/or colonic epithelia, or a level that restores differentiation of tumorigenic cells in these tissues, more preferably a level that causes such intestinal cancerous cells to enter the Death program, allowing them to finally be shed into the lumen of the GI tract. The functional level may be determined by any of the diagnostic methods below. During the course of the prophylaxis or therapy the administered amount of the source of Hedgehog protein may be adjusted based on the Hedgehog protein levels measured in the relevant tissues. The norm for a functional level in a given intestinal tissue in a given physiological condition may be established by determining the Hedgehog proteins levels in the corresponding tissues under comparable conditions in healthy individuals by methods known in the art per se. However, the administered amount of the source of the Hedgehog protein may be therapeutic amount that effects a supranormal level of the Hedgehog protein in GI tract. Such a supranormal level may be a factor 1.5, 2, 3, 5, 10 or higher than the norm for a functional level of Hedgehog protein in GI tract.

In the methods of the invention, the source of Hedgehog protein may be any composition that may administered to a subject, or to organs, tissues or cells, an that is capable of effecting a functional level of Hedgehog protein in the intestinal epithelium. Thus, the source of Hedgehog protein may be a pharmaceutical composition comprising a Hedgehog protein, preferably a pharmaceutical composition that is suitable for oral administration; a gene therapy vector comprising a nucleotide sequence encoding a Hedgehog protein and capable of expression of that sequence in the relevant tissues; an (enteric) bacterium capable of colonising (parts of) the GI tract, wherein the bacterium comprises a nucleotide sequence encoding a Hedgehog protein, that confers to the bacterium the ability to secrete the Hedgehog protein; a (stem) cell, preferably autologous, e.g. an epithelial stem cell or a peripheral mononuclear blood cell that has been transformed ex vivo with a nucleotide sequence that is capable of expressing a Hedgehog protein; a (small) molecule that (up) regulates expression of Hedgehog protein; or a molecule that inhibits Hedgehog protein activity, such as e.g. an antibody against an Hedgehog protein. Suitable sources of Hedgehog protein are described in further detail below.

In a another aspect the invention relates to use of a Hedgehog protein for the manufacture of a pharmaceutical composition for the treatment of a deficiency of a Hedgehog protein in the GI tract. The invention also relates to the use of a gene therapy vector comprising a nucleotide sequence encoding a Hedgehog protein, for the manufacture of a pharmaceutical composition for the treatment of a deficiency of a Hedgehog protein in the GI tract. The invention an enteric bacterium comprising a nucleotide sequence encoding a Hedgehog protein, whereby the nucleotide sequence confers to the bacterium the ability to secrete the Hedgehog protein, for the manufacture of a pharmaceutical composition for the treatment of a deficiency of a Hedgehog protein in the GI tract. In any of these uses the treatment of the deficiency of a Hedgehog protein in the GI tract may be for the prophylaxis of carcinogenesis in the GI tract. Preferably the treatment is for the prophylaxis of gastric or colonic cancer. Any of these uses the treatment of the deficiency of a Hedgehog protein in the GI tract may also be for the treatment of a GI tract carcinoma, preferably for the treatment of gastric or colonic cancer.

In another aspect the invention relates to methods for treating ectopic gastric tissues, such as gastric heterotopia, preferably gastric heterotopia in the esophagus in Meckel's diverticulum, whereby the method comprises reducing the functional level and/or activity of an Hedgehog protein, preferably the Shh protein, in the tissues containing the ectopic gastric tissue. Reduction of the functional level and/or activity of the Hedgehog protein may be achieved by administering a pharmaceutical composition to the GI tract of a subject with ectopic gastric tissue, whereby the pharmaceutical composition comprises a molecule capable of reducing the functional level and/or activity, of the Hedgehog protein. Such a molecule may be an inhibitor of the activity of the Hedgehog protein, such as e.g. an antibody against an Hedgehog protein or against its receptor, or the molecule may be a molecule that reduces or inhibits the expression of the Hedgehog protein, e.g. an antisense nucleic acid at least part of which is complementary to at least a functional part of a Hedgehog mRNA. The complementary part of the antisense nucleic acid is preferably at least 10, 15, 20, 30 or 40 bases long. The functional part of the Hedgehog mRNA preferably comprises 5′ untranslated sequences that are necessary for initiation of translation or it comprises a part of the Hedgehog coding region such that binding of the antisense nucleic acid to the Hedgehog mRNA will block initiation or elongation of translation of the Hedgehog mRNA. Preferably, the complementary part of antisense nucleic acid has less than 40, 25, 10% mismatches with, more preferably the complementary part of the antisense nucleic has no mismatches with the corresponding sequence in the Hedgehog mRNA. Alternatively, the molecule that reduces or inhibits the expression of the Hedgehog protein is a protein or small molecule that interferes with expression of the Hedgehog protein by binding to a functional part of the Hedgehog mRNA, Hedgehog transcription regulatory sequences, or other factors required for the regulation of transcription of the Hedgehog gene.

In yet another aspect, the invention relates to methods for diagnosing the status of a subject with respect to GI tract tumorigenesis, in particular in gastric and colon as well as ectopic tissues. Generally in the diagnosis for GI tract carcinogenesis, morphological markers such as the occurrence of polyps in the colon and gastrum or the morphological distinct markers such as the occurrence of gastric cells ectopically i.e. for instance in the esophagus are used to screen for early onset of GI tract carcinogenesis. In certain cases of early onset to carcinogenesis of the GI tract in particular of gastric, esophagic and colonic tissues, no morphological distinct markers i.e. morphological deviating cells can be identified. The present invention now provides for diagnostic methods in which the level of Hedgehog protein(s) or Hedgehog mRNA are determined in GI tract tissue samples, in particular samples of gastric, esophagic and colonic tissues, whereby a finding of lower than normal levels is indicative for susceptibility for GI tract carcinogenesis, for the progression of the sampled tissues towards the development of a carcinoma or for the presence of a carcinoma. The normal functional level of hedgehog protein in a given intestinal tissue under given physiological condition may be established by determining the Hedgehog proteins levels in the corresponding tissues under comparable conditions in healthy individuals by methods known in the art per se. Methods for determining Hedgehog protein levels in tissue samples preferably use antibodies against Hedgehog proteins. Such methods and antibodies as well as methods for generating anti-Hedgehog (monoclonal) antibodies are provided in the Examples and are generally known in the art (see e.g. Harlow and Lane (1988) Antibodies, A Laboratory Manual, Cold Spring Harbor Laboratory, Cold Spring Harbor Laboratory Press, New York). Similarly, methods and materials for determining the expression of Hedgehog mRNA in tissue sample are provided in the Examples and are generally known in the art (see e.g. Sambrook and Russel (2001) “Molecular Cloning: A Laboratory Manual (3^{rd} edition), Cold Spring Harbor Laboratory, Cold Spring Harbor Laboratory Press, New York).

The invention also provides for novel marker that may be used in the diagnosis of (susceptibility for) ectopic gastric tissue such as gastric heterotopia in the esophagus in Meckel's diverticulum. Those markers comprise next to the previously decried hh markers also Droshophila gene homologues such as human BMP2 and BMP4, of which the elevated levels in the ectopic GI tissues provide an indication that those GI tract tissues are susceptible to carcinogenesis. Thus the invention provide for the diagnosis of ectopic gastric tissue whereby the higher than normal levels of hh, BMP2 and/or BMP4 proteins or mRNAs in a tissue sample are indicative for the presence of ectopic gastric tissues that is susceptible for carcinogenesis. Methods for determining hh, BMP2 and/or BMP4 proteins or mRNAs and for determining the normal levels of these proteins and mRNA's are as described above.

In a further aspect, the invention relates to a gene therapy vector comprising a nucleotide sequence encoding a Hedgehog protein. Nucleotide sequences encoding Hedgehog proteins, gene therapy vectors and methods for their construction and use are as described below.

In another aspect, the present invention relates to an enteric bacterium comprising a nucleotide sequence encoding a Hedgehog protein, whereby the nucleotide sequence confers to the bacterium the ability to secrete the Hedgehog protein. Preferred bacterial hosts are capable of surviving in a mammalian GI tract, preferably capable of colonising a mucosal surface lining of the mammalian GI tract, and preferably, not pathogenic to the mammal in which the bacterial host is to be employed. With respect to the latter aspect it is to be understood that the invention comprises the use of hosts that may normally be pathogenic to the mammal (e.g. Listeria spp., Salmonella spp. or Campylobacter spp.) but that have been modified such that they are no longer pathogenic or virulent. Thus, a preferred microbial host will usually be a non-pathogenic bacterium capable of colonising mucosal surfaces of the mammalian GI tract, which bacterium is transformed with a nucleic acid construct described herein above and below. The microbial host may contain the nucleotide sequence encoding a Hedgehog protein on an episomally replicating molecule, or alternatively and more preferably, integrated into its genome. The latter has the advantage of greater genetic stability. The bacterial host contains the nucleotide sequence encoding a Hedgehog protein as part of an expression construct in which the nucleotide sequence is operably linked to a promoter capable of regulating transcription of the nucleotide sequence. The promoter preferably is active in the host under the conditions that prevail when the bacterium is present in the GI tract of a mammal, more preferably under the conditions that prevail when the host is adhered to the mucosal surfaces of the mammalian GI tract. These may be constitutive promoters but particularly suitable promoters for this purpose are promoters from Gram-positive bacteria that are regulated by cysteine attenuation, such as the promoter of the mapA operon of Lactococcus reuteri (NCBI accession number CAC05301). Particularly preferred is a bacterial host in which the expression construct with nucleotide sequence encoding a Hedgehog protein is integrated into the bacterial genome by means of gene replacement whereby preferably the replaced bacterial gene is a gene that is essential for growth of the bacterium in the environment, e.g. a gene that is required for growth on mineral medium. A preferred bacterial gene that may be used for gene replacement with the Hedgehog expression construct is the thyA gene, which is essential for bacterial growth in the absence of exogenous thymidine (see e.g. Steidler et al., 2000, Science 289: 1352-1355). A preferred host is a bacterium that belongs to a genus selected from the group consisting of Lactobacillus, Lactococcus, Leuconostoc, Streptococcus, Bifidobacterium, Bacteroides, Eubacterium, Clostridiuni, Fusobacterium, Propioizibacterium, Enterococcus, Staphylococcus, Peptostreptococcus, and Escherichia. A further preferred host is a bacterium that is a Lactobacillus or Bifidobacterium species selected from the group consisting of L. reuteri, L. fermentum, L. acidophilus, L. crispatus, L. gasseri, L. johnsonii, L. plantarum, L. paracasei, L. murinus, L. jensenii, L. salivarius, L. minutis, L. brevis, L. gallinarum, L. amylovorus, B. bifidum, B. longum, B. infantis, B. breve, B. adolescente, B. animalis, B. gallinarum, B. magnunm, and B. therinophilum.

Pharmaceutical Compositions

In some methods, Hedgehog protein purified from mammalian, insect or microbial cell cultures, from milk of transgenic mammals or other source is administered in purified form together with a pharmaceutical carrier as a pharmaceutical composition. Methods of producing pharmaceutical compositions comprising Hedgehog proteins are described in are described in U.S. Pat. Nos. 5,789,543 and 6,207,718. The preferred form depends on the intended mode of administration and therapeutic application. The pharmaceutical carrier can be any compatible, nontoxic substance suitable to deliver the polypeptides to the patient. Sterile water, alcohol, fats, waxes, and inert solids may be used as the carrier. Pharmaceutically acceptable adjuvants, buffering agents, dispersing agents, and the like, may also be incorporated into the pharmaceutical compositions.

The concentration of the Hedgehog protein in the pharmaceutical composition can vary widely, i.e., from less than about 0.1% by weight, usually being at least about 1% by weight to as much as 20% by weight or more.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable colour, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

Hedgehog protein is preferably administered parentally. Hedgehog protein for preparations for parental administration must be sterile. Sterilisation is readily accomplished by filtration through sterile filtration membranes, prior to or following lyophilisation and reconstitution. The parental route for Hedgehog protein administration is in accord with known methods, e.g. injection or infusion by intravenous, intraperitoneal, intramuscular, intraarterial or intralesional routes. Hedgehog protein is administered continuously by infusion or by bolus injection. A typical composition for intravenous infusion could be made up to contain 10 to 50 ml of sterile 0.9% NaCl or 5% glucose optionally supplemented with a 20% albumin solution and 1 to 50 μg of the Hedgehog protein. A typical pharmaceutical composition for intramuscular injection would be made up to contain, for example, 1-10 ml of sterile buffered water and 1 to 100 μg of the Hedgehog protein of the present invention. Methods for preparing parenterally administrable compositions are well known in the art and described in more detail in various sources, including, for example, Remington's Pharmaceutical Science (15th ed., Mack Publishing, Easton, Pa., 1980) (incorporated by reference in its entirety for all purposes).

The pharmaceutical compositions of the present invention are usually administered orally. Intradermal, intramuscular or intravenous administration is also possible in some circumstances. The compositions can be administered for prophylactic treatment of individuals suffering from, or susceptible to, carcinogenesis of the GI tract in an amount sufficient to prevent, delay or reduce the severity of subsequent disease. For therapeutic applications, the pharmaceutical compositions are administered to a patient suffering from established disease, carcinogenesis of the GI tract, in an amount sufficient to reduce the severity of symptoms and/or prevent or arrest further development of symptoms. An amount adequate to accomplish this is defined as a “therapeutically-” or “prophylactically-effective dose.” Such effective dosages will depend on the severity of the condition and on the general state of the patient's health.

In the present methods, Hedgehog protein is usually administered at a dosage of about 1 μ/kg patient body weight or more per week to, a patient. Often dosages are greater than 10 μg/kg per week. Dosage regimes can range from 10 μ/kg per week to at least 1 mg/kg per week. Typically dosage regimes are 10 μ/kg per week, 20 μ/kg per week, 30 μ/kg per week, 40 μ/kg week, 60 μg/kg week, 80 μ/kg per week and 120 μg/kg per week. In preferred regimes 10 μ/kg, 20 μ/kg or 40 μ/kg is administered once, twice or three times weekly. Treatment is typically continued for at least 4 weeks, sometimes 24 weeks, and sometimes for the life of the patient. Treatment is preferably administered by oral route. Alternatively, in some conditions it may be desirable to achieve higher than normal levels, e.g. 150% of normal levels, 200% of normal levels or even 300% of normal levels.

Nucleotide Sequences Encoding Hedgehog Proteins

Although the intended use of hh proteins produced by mammalian, insect, or microbial cell culture, or transgenic mammals or alternatively produced in situ in bacteria endogenous i.e. common to the flora of the GI tract is usually administered to humans, the species from which the DNA segment encoding a hh sequence is obtained is not necessarily human. Due to the high percentage of homology between the hh homologues of different species, e.g. human, mouse, Drosophila, zebrafish, and rat (available at the NCBI website: www.ncbi.nlm.nih.gov, under accession numbers: XM_{—}050846, NM_{—}000193, XM_{—}090366, NM_{—}010544, XM_{—}082291, AF124382, and NM_{—}017221) all hh sequences can be used as the provide for the same functionality and are fully interchangeable. The three known hh sequences, i.e. Indian, Sonic and Desert, are equally homologue to such an extent that any hh variant can be applied in the invention. This notion is exemplified by treating colon cancer cells successfully with Shh resulting into differentiation and riddance of the cancerous colon cells, while the natural ligand is in fact Ihh.

The hh DNA sequence was shown to encode precursor hh proteins of consistent amino acids. The entire genomic sequence of hh including Indian, Desert and Sonic of a number of different species including human, mouse, rat, chicken and zebrafish sequences are known and shows a high percentage of homology. Also can be determined whether or not introns are found in the various hh genes, as for instance the human Shh gene of chromosome 7 comprises 2 introns. Transgenic mammals expressing allelic, cognate and induced variants of any of the prototypical sequence described in this reference are included in the invention. Such variants usually show substantial sequence identity at the amino acid level with known hh genes. Such variants usually hybridize to a known gene under stringent conditions or cross-react with antibodies to a polypeptide encoded by one of the known genes. Other examples of genomic and cDNA sequences are available from GenBank or the NCBI website. To the extent that additional cloned sequences of hh genes are required, they may be obtained from genomic or cDNA libraries (preferably human) using known hh sequences. In genomic constructs, it is not necessary to retain all intronic sequences.

For example, some intronic sequences can be removed to obtain a smaller transgene facilitating DNA manipulations and subsequent microinjection. See Archibald et al., WO 90/05188 (incorporated by reference in its entirety for all purposes). Removal of some introns is also useful in some instances to enhance expression levels. Removal of one or more introns to reduce expression levels to ensure that posttranslational modification is substantially complete may also be desirable. It is also possible to delete some or all of the non-coding exons. In some transgenes, selected nucleotides in hh encoding sequences are mutated to remove proteolytic cleavage sites. The sequence encoding a hh protein or any functional homologue thereof may be introduced in an enteric bacterium as described below, or may be incorporated in a viral or non-viral gene therapy vector as described below or introduced as naked DNA expression construct.

Nucleotide sequences encoding Hedgehog proteins may also be defined by their capability to hybridise with the nucleotide sequences encoding the amino acid sequences of SEQ ID NO. 1 to SEQ ID NO. 3, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

Recombinant Techniques and Methods for Recombinant Production of Hedgehog (Poly)Peptides

Peptides and polypeptides for use in the present invention, such e.g. the Hedgehog proteins, can be prepared using recombinant techniques in which a nucleotide sequence encoding the polypeptide of interest is expressed in cultured cells such as described in Ausubel et al., “Current Protocols in Molecular Biology”, Greene Publishing and Wiley-Interscience, New York (1987) and in Sambrook and Russel (2001) “Molecular Cloning: A Laboratory Manual (3^{rd} edition), Cold Spring Harbor Laboratory, Cold Spring Harbor Laboratory Press, New York; both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis). More specifically, methods for recombinant production of Hedgehog proteins are described in U.S. Pat. No. 5,789,543.

Typically, nucleic acids encoding the desired polypeptides are used in expression vectors. The phrase “expression vector” generally refers to nucleotide sequences that are capable of affecting expression of a gene in hosts compatible with such sequences. These expression vectors typically include at least suitable promoter sequences and optionally, transcription termination signals. Additional factors necessary or helpful in effecting expression can also be used as described herein. DNA encoding a polypeptide is incorporated into DNA constructs capable of introduction into and expression in an in vitro cell culture. Specifically, DNA constructs are suitable for replication in a prokaryotic host, such as bacteria, e.g., E. coli, or can be introduced into a cultured mammalian, plant, insect, e.g., Sf9, yeast, fungi or other eukaryotic cell lines. Expression constructs may also be used to generate transgenic plant or transgenic animals capable of producing the protein of interest. Alternatively, both viral and non-viral expression constructs may be employed for gene therapy as outlined below.

DNA constructs prepared for introduction into a particular host typically include a replication system recognized by the host, the intended DNA segment encoding the desired polypeptide, and transcriptional and translational initiation and termination regulatory sequences operably linked to the polypeptide encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof.

The selection of an appropriate promoter sequence generally depends upon the host cell selected for the expression of the DNA segment. Examples of suitable promoter sequences include prokaryotic, and eukaryotic promoters well-known in the art (see, e.g. Sambrook and Russel, 2001, supra). The transcriptional regulatory sequences typically include a heterologous enhancer or promoter that is recognized by the host. The selection of an appropriate promoter depends upon the host, but promoters such as the trp, lac and phage promoters, tRNA promoters and glycolytic enzyme promoters are known and available (see, e.g. Sambrook and Russel, 2001, supra). Expression vectors include the replication system and transcriptional and translational regulatory sequences together with the insertion site for the polypeptide encoding segment can be employed. Examples of workable combinations of cell lines and expression vectors are described in Sambrook and Russel (2001, supra) and in Metzger et al. (1988) Nature 334: 31-36; For example, suitable expression vectors can be expressed in, e.g., insect cells, e.g., Sf9 cells, mammalian cells, e.g., CHO cells and bacterial cells, e.g., E. coli.

In vitro mutagenesis and expression of mutant proteins are described generally in Ausubel et al. (1987, supra) and in Sambrook and Russel (2001, supra). Also see, Kunkel (1985, supra; describing site directed mutagenesis) and Roberts et al. (1987, supra; describing cassette mutagenesis).

Another method for preparing polypeptides is to employ an in vitro transcription/translation system. DNA encoding a polypeptide is cloned into an expression vector as described supra. The expression vector is then transcribed and translated in vitro. The translation product can be used directly or first purified. Polypeptides resulting from in vitro translation typically do not contain the post-translation modifications present on polypeptides synthesized in vivo. Methods for synthesis of polypeptides by in vitro translation are described by, for example, Berger & Kimmel, Methods in Enzymology, Volume 152, Guide to Molecular Cloning Techniques, Academic Press, Inc., San Diego, Calif., 1987 (incorporated herein by reference in its entirety)

Gene Therapy Constructs

Viral and or non-viral vectors (or constructs) are used for transfecting the targeted GI tract tissue in particular the gastric and or colonic tissues. The term vector refers to a nucleic acid, protein, lipid or other molecule capable of transporting a nucleic acid to which it has been operatively linked. Vectors may include circular double stranded DNA plasmids and viral vectors. Some vectors are capable of autonomous replication in a host cell into which they are introduced (such as bacterial vectors having a bacterial origin of replication and episomal mammalian vectors). Other vectors (such as non-episomal mammalian vectors) may be integrated into the genome of a host upon introduction into the host cell, and thereby may be replicated along with the host genome. Certain vectors may be capable of directing the expression of genes to which they are operatively linked. In recombinant vectors of the invention, the nucleotide sequences encoding a peptide may be operatively linked to one or more regulatory sequences, selected on the basis of the host cells to be used for expression. The terms operatively or operably linked mean that sequences encoding the peptide are linked to the regulatory sequence(s) in a manner that allows for expression of the peptide compound. The term regulatory sequence includes promoters, enhancers, polyadenylation signals and other expression control elements. Such regulatory elements are described in for example in Goeddel; Gene Expression Technology: Methods in Enzymology 185, Academic Press, San Diego, Calif. (1990). Recombinant expression vectors of the invention may be designed for expression of the HH peptide in prokaryotic or eukaryotic cells. For example, HH peptides may be expressed in bacterial cells such as E. Coli, insect cells (using baculovirus expression vectors) yeast cells or mammalian cells. Suitable host cells are discussed further in Goeddel; Gene Expression Technology: Methods in Enzymology 185, Academic Press, San Diego, Calif. (1990). Alternatively, the recombinant hh expression vector may be transcribed and translated in vivo, for example using T7 promoter regulatory sequences and T7 polymerase. Examples of vectors for expression in yeast S. cerevisiae include pYepSec1, pMFa and pYES2. Examples of Baculovirus include pAc series and the pVL series. Mammalian expression vectors include pCDM8, often control functions are provided by viral regulatory elements. For example commonly used promoters are derived from polyoma, Adenovirus 2, cytomegalovirus and Simian Virus 40. Vector DNA can be introduced into the prokaryotic or eukaryotic cells via conventional transformation or transfection techniques, including introducing foreign nucleic acid into a host cell using calcium phosphate or calcium chloride co-precipitation, DEA-dextran mediated transfection, lipofection, electroporation, microinjection and viral mediated transfection of which examples can be found in Sambrook et al. (Molecular Cloning: A Laboratory Manual, 2^{nd} Edition, Cold Spring Harbor Laboratory press (1989), and other laboratory manuals. Methods for introducing DNA into mammalian cells in vivo are also known, and may be used to deliver the vector DNA of the invention to a patient for gene therapy. A nucleic acid sequence of the invention may be delivered to cells in vivo using methods such as direct injection of DNA, receptor-mediated transfection or non-viral transfection and lipid based transfection, all of which may involve the use a gene therapy vectors. Defective retroviruses are well characterised for use as gene therapy vectors (Review by Miller, A. D. (1990) Blood 76:271). Protocols for producing recombinant retroviruses and for infecting cells in vitro or in vivo with such viruses can be found in Current Protocols in Molecular Biology, Ausubel, F. M. et al (eds.) Greene Publishing Associates, (1989), Sections 9.10-9.14 and other standard laboratory manuals. Examples include pLJ, pZIP, pWE and pEM, which are well known to those skilled in the art. (See for example Patent applications WO94/26914 and WO95/02697). For use as a gene therapy vector, the genome of an adenovirus may be manipulated so that it encodes and expresses a hh peptide of the invention, but is inactivated in terms of its availability to replicate in a normal lytic viral life cycle. See for example Berkner et al (1988) Biotechniques 6:616, Rosenfeld et al. (1991) Science 252:431-434 and Rosenfeld et al (1992) Cell 68:143-155. Suitable adenoviral vectors derived from the adenovirus strain Ad type 5 d1234 or other strains of adenovirus (e.g. Ad2, Ad3, Ad7 etc.) are well known to those skilled in the art. Recombinant adenoviruses are advantageous, as they do not require dividing cells to be effective gene delivery vehicles and can be used to infect a wide variety of cell types, including epithelium, endothelial, hepatocytes and muscle cells. Adeno-associated virus (AAV) may be used as a gene therapy vector for delivery of DNA for gene therapy purposes. AAV may be used to integrate DNA into non-dividing cells (see for example Flofte et al. (1992) Am. J. Respir. Cell. Mol. Bio. 7:349-356; Samulski et al. (1989) J. Virol. 63:3822-3828 and Patent applications WO91/18088; WO93/09239; U.S. Pat. No. 4,797,368; U.S. Pat. No. 5,139,941 and EP 488 528. An AAV vector may be used to introduce DNA into cells (see for example Hermonat et al.(19⁸5) Mol. Cell. Biol. 4:2072-2081. Herpes viral and or Lenti viral gene therapy may also be adapted for use in the invention. General methods for gene therapy are known in the art. See for example U.S. Pat. No. 5,399,346 by Anderson et al (incorporated herein by reference).

### DESCRIPTION OF THE FIGURES

FIG. 1: Ihh is expressed by terminally differentiated enterocytes

(A) In situ hybridisation using an Ihh probe on normal human colon, detection with purple AP Substrate. The terminally differentiated enterocytes at the tips of the crypts (arrow) produce Ihh mRNA. (B,C) Immunohistocheristry on human (B) and rat (C) adult colon using the anti-Ihh antibody and DAB detection. Ihh protein is expressed by the terminally differentiated enterocytes in both species (arrows). (D) Western blot showing expression of Villin, Ihh and loading control β-Actin in butyrate treated HT-29 cells. Ihh expression is induced as the HT-29 cell differentiates. Original magnification: A-C: 80×.

FIG. 2: Expression of Ptc, BMP2, BMP4, BNF3b and Engrailed-1

Immunohistochemistry on normal rat colon, using DAB as a substrate. Expression of (A) the Hh receptor Ptc is detected in the epithelial cells throughout the crypt and in several stromal cell types (arrows). (B) BMP2 is expressed by the terminally differentiated enterocytes (arrow). (C,D) Myofibroblast-like cells (arrow, C) and some epithelial cells with endocrine cell morphology (arrow, D) express BMP4. (E) The transcription factor HNF3β is detected at highest levels in the nuclei of the epithelial cells at the base of the crypt and (F) in some lamina propria lymphocytes. (G and H) A similar expression pattern was found for Engrailed-1. Original magnification: A,B: 80×; C: 200×; D: 80×; E: 100×; F,G: 60×; H: 100×.

FIG. 3 : The effect of cyclopamine treatment on the expression of putative Hh targets

(A) Western blots showing protein levels of putative Hedgehog regulated proteins. The first seven lanes represent colonic homogenates of seven individual control animals whereas the seven lanes on the right are cyclopamine treated animals. The molecular weight is indicated in kDa on the right of each blot. (B) Quantification of blots shown in (A), mean and standard error of the relative expression compared to the mean of the seven controls. p values (student's t-test): Ihh, P=0.08; BMP2, P=0.25; BMP4, P=0.0001; HNF3b, P=0.001; En-1, P=0.002; GATA6, P=0.005.

FIG. 4: The effect of cyclopamine treatment on proliferation and differentiation

(A) Western blots showing protein levels of markers of differentiation and proliferation. The first seven lanes represent colonic homogenates of seven individual control animals whereas the seven lanes on the right are cyclopamine treated animals. The molecular weight is indicated in kDa on the right of each blot. (B) Quantification of blots shown in (A), mean and standard error of the relative expression compared to the mean of the seven controls. P values: Villin, P=0.02; ITF, P=0.008; Cyclin D1 P=0.01; PCNA, P<0.0001. (C) Graph showing the number of BrdU labelled cells per crypt in controls and cyclopamine treated animals. Student's t-test: P=0.036.

FIG. 5: Loss of Ihh precedes morphological change in the adenoma carcinoma sequence

(A-E) Immunohistochemical detection of Ihh in human resection specimens of sporadic adenomas. (A) A sporadic adenoma (asterisks) bordering a patch of histologically normal epithelium (arrows). (B) blow-up of the area boxed with the continuous line in (A), the terminally differentiated histologically normal epithelial cells show intense staining with the anti-Ihh antibody. (C) No staining in the superficial epithelial cells of the adenoma boxed with the dotted line in (A). (D) Another example of Ihh expression in normal tissue (arrow) and (E) loss of Ihh expression in the adenomatous area of a sporadic adenoma. (F-J) Immunohistochemical detection of Thh and β-catenin in a resection specimen of a patient with FAP. (F) Ihh stain. Loss of Ihh expression in a few morphologically normal crypts (asterisks) with a sharp transition to normal Ihh expression in the adjacent crypts on both sides (arrows). (G) β-catenin stain in adjacent slide. The same crypts show abnormal localisation of β-catenin (see blow-ups). (E) Blow-up of boxed area in F. A sharp transition (arrow) can be seen between the crypt with normal Ihh expression and the crypt with loss of Ihh expression. (I) Loss of membrane staining and cytoplasmic accumulation (arrows) of β-catenin in the area boxed with the dotted line in (G). (J) Normal strong membrane staining (arrows) in the epithelial cells from the adjacent tissue boxed with the continuous line in (G). Original magnifications: A: 120×; B, C: 800×; D, E: 300×; F, G 500×; H-J 1000×.

FIG. 6: Exogenous Hh protein restores differentiation in HT-29 cells

Western blot showing villin expression in duplicate cultures of HT-29 cells grown in a monolayer in the presence or absence of recombinant Shh for 48 hours, using two cultures with butyrate as positive control. The graph depicts mean and standard error of the relative villin expression versus control cultures in two independent experiments (n=4 per condition). Data were analysed by one-way ANOVA and Tukey's post hoc correction. Shh versus control: P<0.01; butyrate versus control: P<0.05.

FIG. 7

Shh mRNA expression along the human GI tract. Shh expression is expressed in the fundic glandular region (B, arrow) of the adult stomach. Shh is not detected in the esophagus (A). Minimal staining is evident in the antrum of the stomach (C), duodenum (D), and at the base of villi in the small intestine (E, arrow at base). Shh signal in colon (F) is strongest at the base of crypts (arrow).

FIG. 8

Shh protein expression along the human and murine GI tract. (A-G) Sections of the human GI tract, Immunohistochemical staining with an antibody against the Shh precursor protein. We found intense staining in the fundic glands (B) whereas no staining was found in esophagus (A), antrum (C), Brunner's glands (D), duodenum (E), ileum (F) or colon (G). Sections of the murine GI tract (H-N) give the same results. Shown are: forestomach (H), fundic glands (I), antral glands (J), duodenum (K), jejunum (L), ileum (M), and colon (N).

FIG. 9

Shh expression is lost in intestinal metaplasia of the stomach. Immunohistochemical triple stain of Shh (blue) MUC5AC (red) and MUC2 (brown). (A-C) Specimens of two different patients with intestinal metaplasia. (A) This specimen shows normal glands with MUC5AC expressing pit cells (arrowhead, red stain), Shh expressing gland cells (arrow, blue stain) and adjacent metaplastic glands with MUC2 expressing goblet cells (asterisks, brown stain). (B) Blow-up of boxed area in A, note the MUC5AC expression by goblet cells around the pit-gland transition. (C) Another example of metaplastic gland (asterisks) amidst normal Shh expressing glands (arrow). (D) Although most cases showed replacement of only the glands or both pits and glands by intestinal cells, in this patient we observed a mix of intestinal MUC2 expressing goblet cells (brown) and gastric MUC5AC expressing pit cells (red) in the pit region. (E-G) Three types of goblet cells were observed in this study, goblet cells that express exclusively MUC2 (arrow in E), goblet cells that co-express MUC2 and MUC5AC (arrow in F), and goblet cells that express only MUC5AC (arrow in G).

FIG. 10

Shh is expressed in fundic gland ectopies. (A-C) Three different cases of Meckel's diverticulum. (A) No Shh is detected in a Meckel's specimen with purely intestinal histology. (B) Shh staining (brown precipitate, arrow) in a gastric fundic gland adjacent to a region of goblet cell containing intestinal histology (asterisks). (C) Double stain of the gastric mucin MUC5AC (blue, arrow) and Shh (brown, asterisks) in a Meckel's diverticulum with fundic gland histology. (D,E) Esophageal metaplasias. (D) No Shh is detected in the intestinal metaplasia of Barrett's mucosa. (E) A case of fundic gland metaplasia of the esophagus with Shh expressing cells (brown precipitate, asterisks).

FIG. 11

Cyclopamine treatment disturbs enterocyte maturation. (A-D) H&E stain of distal colon from control (a,c) and cyclopamine treated (b,d) animals. (C) blow-up of boxed area in A, arrows denote normal slender terminally differentiated enterocytes in controls. (D) blow-up of boxed area in B, arrows denote two enterocytes with normal appearance amid abnormal enterocytes with enlarged nuclei. (E,F) Villin immunohistochemistry. (E) Normal enterocytes show light cytoplasmic staining and strong staining of the apical membrane (arrows). (F) In the abnormal appearing enterocytes of cyclopamine treated animals apical staining is diminished and cytoplasmic staining enhanced (arrows). (G,H) CA IV immunohistochemistry. (G) Strong staining of the enterocyte apical membrane in control animals (arrows). (H) Loss of carbonic anhydrase (CA) IV expression in the abnormal enterocytes in cyclopamine treated animals, arrows denote a few remaining CA IV expressing enterocytes. (I-L) ITF immunohistochemistry. (K) blow-up of boxed area in I, strong staining of goblet cells (asterisks) and apical staining of enterocytes (arrow) in control colon. (L) blow-up of boxed area in J, strong immunoreactivity of the abnormal enterocytes (arrows) in cyclopaamine treated animals.

FIG. 12

The effect of cyclopamine treatment on proliferation in the adult rat colon (A) Western blots showing protein levels of markers of proliferation. The first seven lanes represent colonic homogenates of seven individual control animals whereas the seven lanes on the right are cyclopamine treated animals. (B) Quantification of blots (c) Graph showing the number of BrdU labelled cells per crypt in controls and cyclopamine treated animals.

FIG. 13

Western blots of control HT-29 cells and HT-29 cells treated for 24 hours. (B) Mean and standard error of 3 independent experiments (co=control, but=butyrate, cyc=cyclopamine). (C) Western blots of HT-29 cells treated with 2.5 μg/ml recombinant Shh for the indicated periods.

TABLE 1
The Antibodies used in this study.

| | Name/ | | | | |
|---|---|---|---|---|---|
| Antigen | clone | Source | Provenance | IHC | WB |
| Ihh | I-19 | Goat | Santa Cruz | 1:50 | 1:500 |
| Ptc | C-20 | Goat | Santa Cruz | 1:50 | |
| Ptc | | Rabbit | Dr R. Töftgard | 1:200 | |
| βActin | I-19 | Goat | Santa Cruz | | 1:2000 |
| GATA-6 | H-29 | Rabbit | Santa Cruz | 1:50 | 1:500 |
| Villin | C-19 | Goat | Santa Cruz | | 1:1000 |
| BMP2 | mAb 355 | mAb | R&D systems | 1:1000 | 1:5000 |
| BMP4 | mAB 757 | mAb | R&D systems | 1:50 | 1:1000 |
| Cyclin D1 | DCS6 | mAb | Neomarkers | | 1:500 |
| En-1 | 4G11 | mAb | DSHB, | 1:25 | 1:500 |
| | | | Dr J. M. Jessell | | |
| HNF3β | 4C7 | mAb | DSHB, | 1:10 | 1:500 |
| | | | Dr J. M. Jessell | | |
| β-catenin | 14 | mAb | Transduction | 1:1000 | |
| | | | laboratories | | |
| ITF | | Rabbit | Dr D. K. Podolsky | | 1:500 |

Commercial antibodies were obtained from Santa Cruz (Santa Cruz, CA), R&D systems (Minneapolis, MN), Neomarkers (Fremont, CA) and Transduction laboratories (Lexington, KY). The antibodies developed by Dr J. M. Jessell's lab, were obtained from the Developmental Studies Hybridoma Bank (DSHB, Iowa City, IA). IHC = immunohistochemistry, WB = Western blot, mAb = monoclonal antibody.

### EXAMPLES

### Materials and Methods

A. Antibodies

Antibodies used are listed below; concentrations for immunohistochemistry are in normal font those used for immunoblot are italicised. An anti-BMP2 mouse monoclonal antibody (mAb) (355; 1:1000; 1:2000) and an anti-BMP4 mAb (757; 1:500; 1:2000) were from R&D systems (Minneapolis, Minn.). A goat polyclonal anti-Shh (N-19) that recognises the Shh precursor protein (van den Brink et al., 2001) (1:200; 1:2000), a goat polyclonal anti-Ihh (I-19; 1:50; 1:500), a goat polyclonal anti-Ptc (C-20, 1:50), a Rabbit polyclonal anti-GATA6 (H-29; 1:50; 1:500), a goat polyclonal anti-Villin (C-19; 1:1000) and a goat polyclonal anti-a-actin (I-19; 1:1000) were all from Santa Cruz (Santa Cruz, Calif.). An anti-HNF3b mAb (4C7; 1:10; 1:1000), and an anti-Engrailed-1 mAb, both developed by Dr J. M. Jessell's laboratory, were obtained from the Developmental Studies Hybridoma Bank (Iowa City, Iowa). An anti-PCNA mAb (1:5000) and an anti-BrdU (1:100) were from Roche (Almere, the Netherlands). Specificity of all antibodies used in immunohistochemistry was confirmed on immunoblot. Secondary antibodies used were all from Dako (Glostrup, Denmark). An anti-cyclin D1 mAb (DCS6) was from Neomarkers (Fremont, Calif.). An anti-b-catenin mAb (clone 14) was from Transduction laboratories (Lexington, Ky.). A rabbit polyclonal anti-Ptc (1:200) and a rabbit polyclonal anti-ITF were a gift of Dr R. Toftgard and Dr D. K. Podolsky respectively.

For purposes of experimentation on gastric tissues a goat polyclonal α-Shh antibody (N-19, 1:250) produced by immunising with an amino acid sequence mapping at the amino terminus of the murine Shh precursor was obtained from Santa Cruz Biotechnology (Santa Cruz, Calif.). We have shown previously that this antibody specifically recognises the 49-kDa Shh precursor protein (van den Brink et al., 2001). A mouse monoclonal α-H⁺/K⁺-ATPase (1:6000) was from Affinity Bioreagents (Golden, Colo.). A mouse monoclonal anti-MUC5AC (1:50, clone 45M1) was from Lab Vision (Fremont, Calif.). A mouse monoclonal anti-MUC2 (1:100, clone CCP58) was from Novocastra (Newcastle upon Tyne, England).

B. Immunohistochemistry and In Situ Hybridisation

Formalin fixed paraffin embedded human biopsy and resection specimens of uninflamed colonic mucosa, sporadic adenomas, sporadic carcinomas and adenomas from patients with FAP were obtained from the archives of the pathology department of the Academic Medical Center following institutional standards for human subject research. Immunohistochemistry was performed on 4μ sections using a three-step diaminobenzidine (DAB) detection method with antigen retrieval as described in detail previously (van den Brink et al., 2000). For BrdU visualisation, sections were incubated in 2N HCl at 37° C. for 60 minutes after deparaffinization and then washed in boric acid pH 8.5. Sections were counterstained with Mayer's hematoxylin, except when stained for HNF3β, engrailed-1 or β-catenin to allow optimal visualisation of nuclear staining. BrdU positive nuclei were scored as described (van den Brink et al., 2001).

In situ hybridisation on paraffin sections was performed using digoxigenin-labeled mRNA probes for human Shh and Ihh, a gift of Dr C. Tabin.

For purposes of experimentation on gastric tissues the methods used for staining of a single epitope on paraffin sections have been described in detail previously (van den Brink et al., 2000). For double staining of Shh and MUC5AC sections were incubated with a mixture of the anti-Shh and anti-MUC5AC overnight. The following day sections were incubated with a mixture of HRP coupled rabbit anti-goat Ig (1:100, Dako) and biotinylated rabbit anti-mouse Ig (1:250). First the HRP was detected with fast DAB as described (Tytgat et al, 1994) and hereafter sections were incubated with streptavidin β-galactosidase (strep β-gal, 1:50 in PBS, Roche, Almere, The Netherlands) for 30 min at RT. The β-gal was detected with 40 μg/ml X-Gal (Gibco, Breda, the Netherlands) in iron phosphate buffer (0.02% MgCl₂.6H2O, 0.099% potassium ferricyanide, 0.127% potassium ferrocyanide) at 37° C. for 15 min, resulting in a turquoise color.

Simultaneous immunohistochemical detection of three different epitopes was performed largely as described previously (van den Brink et al., 2000). For triple staining of Shh, MUC5AC and MUC2 the following protocol was developed. Sections were rehydrated and blocked as described and incubated with the anti-MUC5AC monoclonal overnight. The following day sections were incubated with an alkaline phosphatase (AP) coupled goat anti-mouse Ig (1:20, Dako) in PBS containing 10% human AB serum for one hour. After washing in Tris Buffered Saline, AP activity was detected using Fast Red (Dako) resulting in a red precipitate. Hereafter sections were heated to 100° C. for 5 min to remove antibodies and enhance antigen retrieval for the anti-Shh antibody. Sections were blocked and incubated with a mixture of monoclonal anti-Muc2 and goat polyclonal anti-Shh overnight. On day 3 sections were incubated with a mixture of rabbit anti-mouse-HRP (1:50) and rabbit anti-goat-biotin (1:200) in PBS containing 10% human AB serum for one hour. First the HRP was detected with DAB as described above and hereafter sections were incubated with strep β-gal for 30 min at RT. The β-gal was detected as above.

C. Immunoblot

The distal half of the rat colon was dissected along the longitudinal axis and one half was homogenised and processed for Western blotting as described below. Murine stomach and small intestine were homogenised in lysis buffer (300 mmol/L NaCl, 30 mmol/L Tris, 2 mmol/L MgCl₂, 2 mmol/L CaCl₂, 1% Triton X-100, pH 7.4, supplemented with 1 tablet of protease inhibitor [Roche] per 50 ml). Protein concentration was measured using the Bradford method. Lysates were diluted 1:3 in protein sample buffer (125 mmol/L Tris/HCl, pH 6.8; 4% sodium dodecyl sulfate; 2% β-mercaptoethanol; 20% glycerol, 1 mg bromophenol blue), and 100-200 μof homogenate was loaded per lane on a sodium dodecyl sulfate-pol-yacrylamide gel electrophoresis gel. After protein separation, the proteins were blotted on to a PVDF membrane (Millipore, Bedford, Mass.). Membranes were blocked with 2% protifar (Nutricia, Zoetermeer, The Netherlands) in phosphate-buffered saline (PBS), supplemented with 0.1% Tween-20 for 1 hour at room temperature. After a brief wash in washing buffer (0.2% protifar; 0.1% Tween-20), membranes were incubated overnight at 4° C. with antibody diluted in washing buffer at the indicated concentration. The next day, membranes were washed 3 times for 5 minutes each and subsequently incubated with a secondary horseradish peroxidase tHRP)-conjugated antibody in a 1:2000 dilution. After enhanced chemoluminescence using Lumilight⁺ substrate (Roche, Mannheim, Germany), antibody binding was visualised using a Lumi-Imager (Boehringer Mannheim, Mannheim, Germany).

D. Cyclopamine Treatment

To assess a possible role of hedgehog signaling in the life cycle of gastric epithelial cells, mice were treated with daily injections of cyclopamine, a potent hedgehog signaling inhibitor (Incardona et al., 1998; Taipale et al., 2000; Bitgood et al.,) that inhibits hedgehog signaling somewhere downstream of patched and upstream of the transcriptional effectors of the Gli family, most likely at the level of smoothened. The cyclopamine was a kind gift of Dr. W. Gaffield. The study protocol was approved by the animal ethics review board of the University of Amsterdam. Cyclopamine was administered complexed with 2-hydroxypropyl-β-cyclodextrin (HBC; Sigma). A cyclopamine-HBC stock solution was produced by suspending 1 mg cyclopamine per milliliter of 45% HBC in sterile PBS and stirring for 60 minutes at 65° C. The cyclopamine-HBC was stored at −20° C. until administration. Eight 7-week-old female C57BL/6 mice were given daily intraperitoneal injections of 2 mg/kg cyclopamine-HBC for 14 days. Eight mice received solvent only as a control. After 14 days, mice were given a single intraperitoneal injection of 150 mg/kg BrdU to label cells in S phase. One hour after BrdU administration, mice were killed by cervical dislocation. To allow optimal orientation of the gastric tissue, flat stomachs were prepared according to the method described by Lee et al. and fixed in this position with needles. The stomach was transected along the longitudinal axis, half of the stomach was homogenized, and gastric lysates were produced as described above. The other half of the stomach and the small intestine were then fixed in 4% paraformaldehyde and embedded in paraffin. To assess proliferation of gastrointestinal epithelial cells in the cyclopamine-treated mice, gastric and small intestinal specimens were stained with antibodies against BrdU and PCNA. Two pictures of each section were taken at 100× magnification, and positive nuclei were counted by investigators who were blind to the treatment in each microscope field with the use of an image analysis program (EFM Software, Rotterdam, The Netherlands). In each field, 5 well-oriented vertical units were counted for the PCNA stain and 10 for the BrdU stain (BrdU staining requires more counted crypts because of the low amount of BrdU-labeled cells). The average numbers of positive nuclei per vertical unit were compared between groups. To enable comparison of the results between animals, all sections visualized the entire axis from the superficial epithelium to the muscularismucosa.

In addition to the mice described above, 8-week-old female Wistar rats (n=7) were treated with daily intraperitoneal injections of 1 mg/kg of the Hedgehog inhibitor cyclopamine complexed with 2-hydroxypropyl-β-cyclodextrin (HBC; Sigma) as described (van den Brink et al., 2001). Control rats (n=7) received solvent only. After 14 days, rats were given a single intraperitoneal injection of 150 mg/kg BrdU one hour before being killed.

E. Cell Culture

HT 29 colon cancer cell lines were cultured according to routine procedures in the presence of 10% fetal calf serum (GIBCO). Recombinant Shh (R&D systems) was used at the indicated concentrations; Butyrate (Sigma) was used at a 5 mM concentration.

F. Tissues

The distal half of the rat colon was dissected along the longitudinal axis and one half was homogenised and processed for Western blotting as described (van den Brink et al., 2001). To study Shh expression in the human GI tract we used specimens from the archives of the department of pathology of the Academic Medical Center and the Department of Pathology of the Massachusetts General Hospital and Brigham and Women's Hospital. We examined tissue with normal histology of at least 6 different patients to investigate each site along the normal GI tract for Shh expression. Other specimens included: tissue from 16 different patients with intestinal metaplasia of the stomach, 13 resection specimens of Meckel's diverticulum and 6 resection specimens of patients with Barrett's esophagus.

G. In Situ Hybridisation

Human Shh cDNA, a gift of Dr C. Tabin, was used to transcribe a digoxigenin-labeled (Roche, Mannheim, Germany) mRNA probe. Paraffin sections (4-6 μm) of archival human tissue were used for in situ hybridisation using previously published methods (Robetrs et al., 1998)

### Example 1

### Colonic Tissues

1.1. Ihh is Expressed in the Adult Colon and Regulates the Expression of BMP-4 and Transcription Factors Involved in Tissue Specific Gene Expression

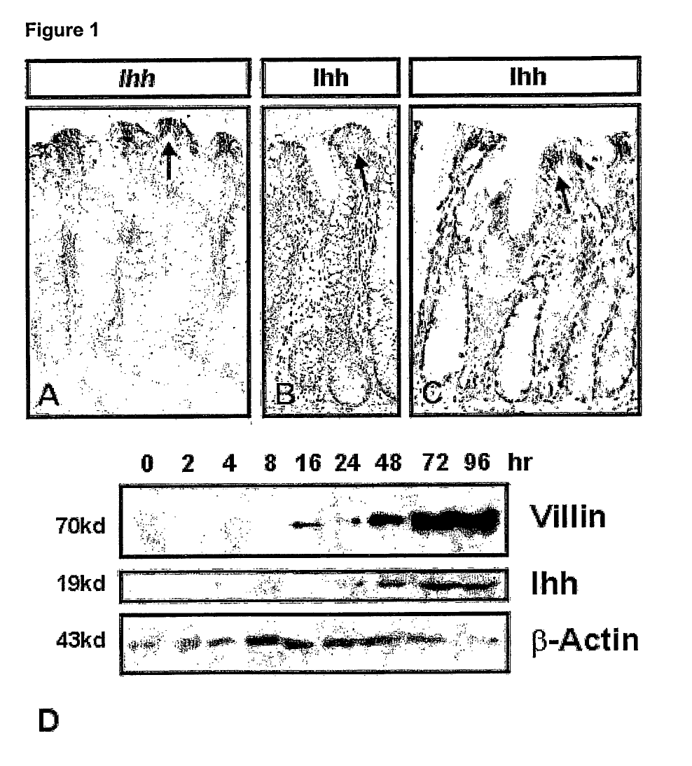

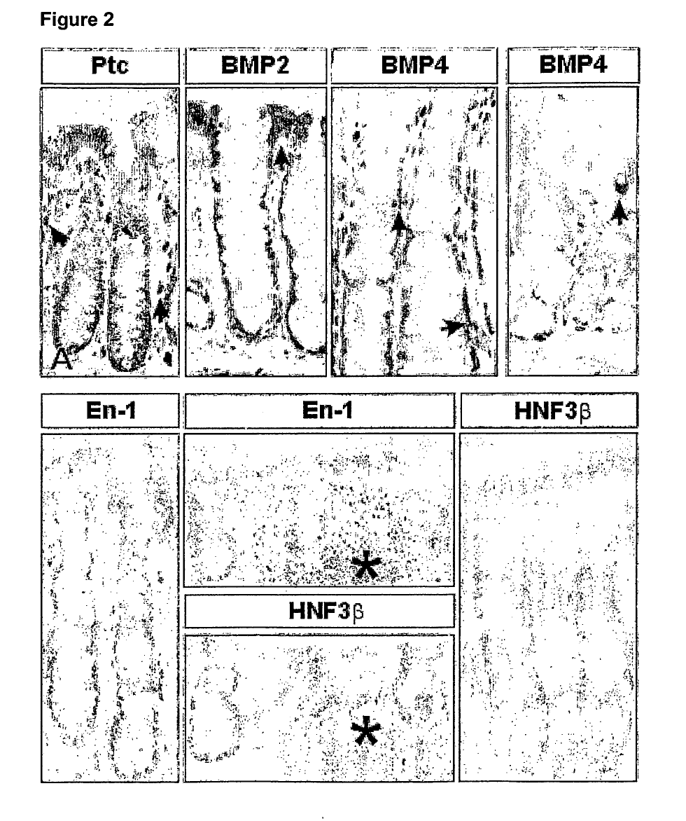

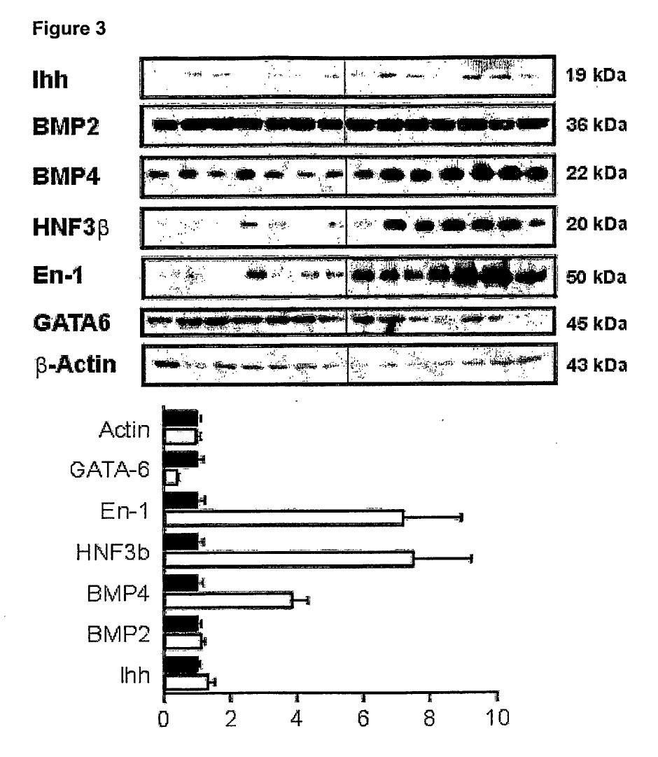

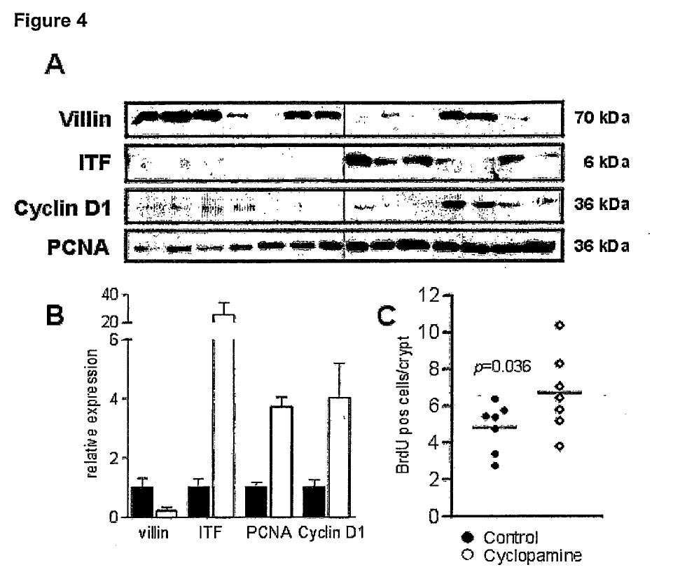

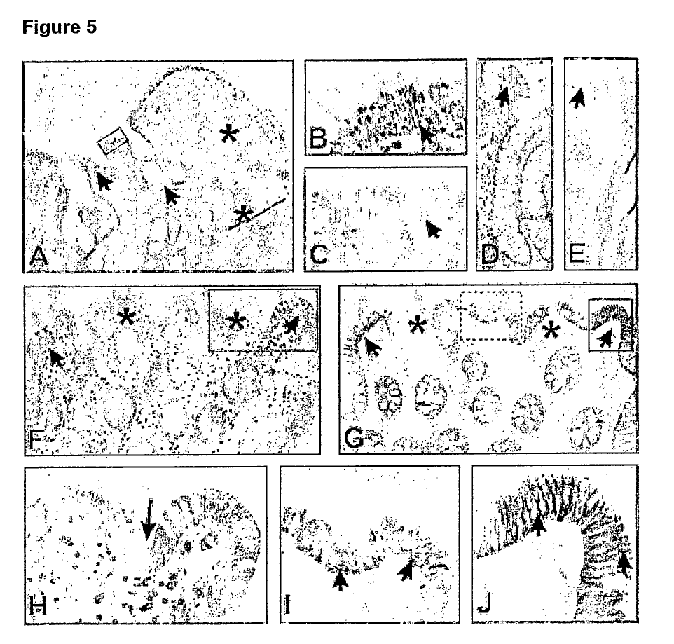

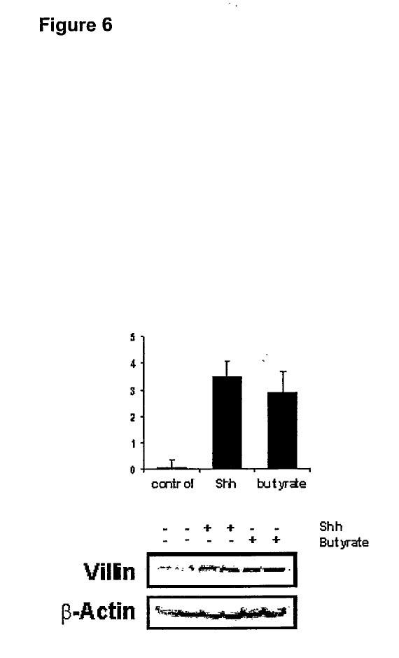

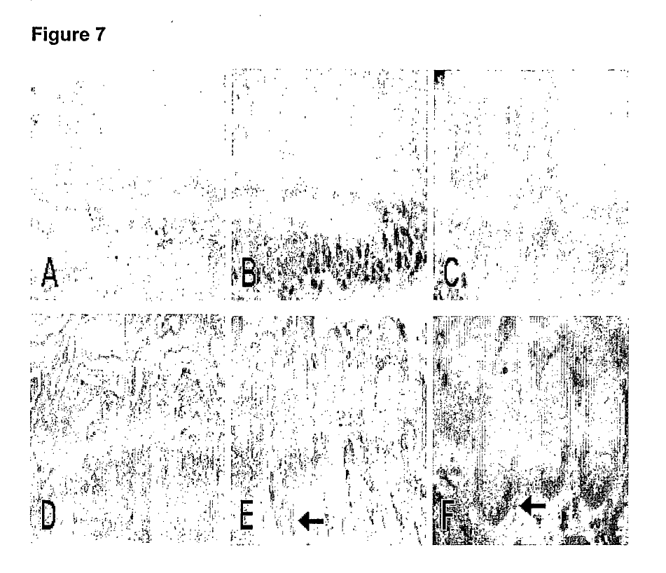

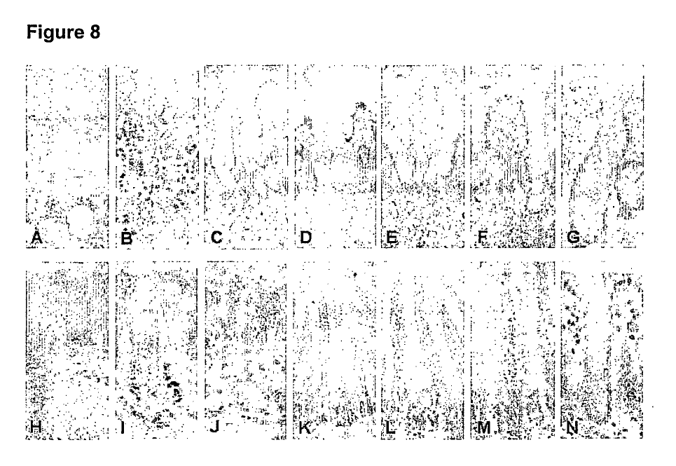

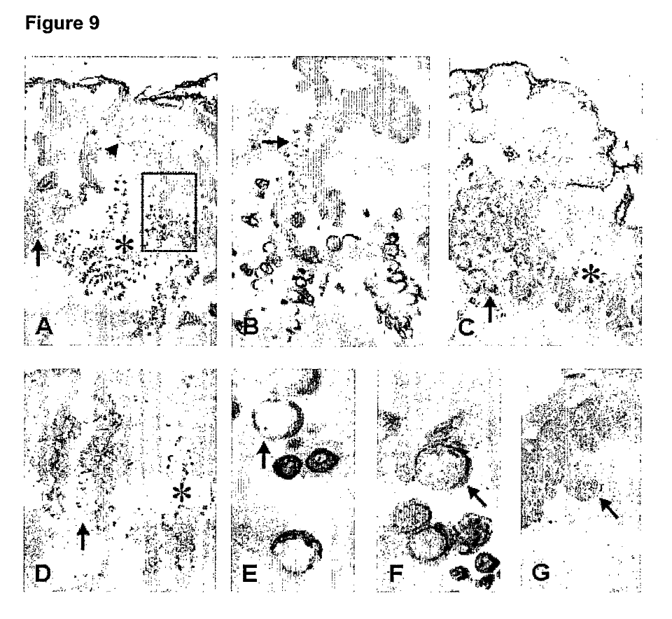

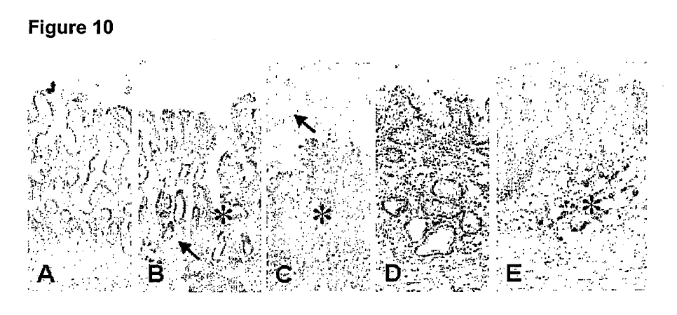

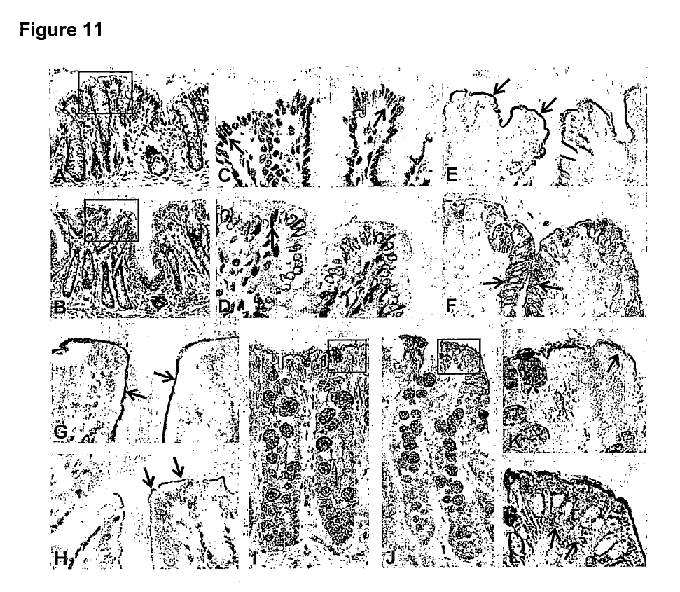

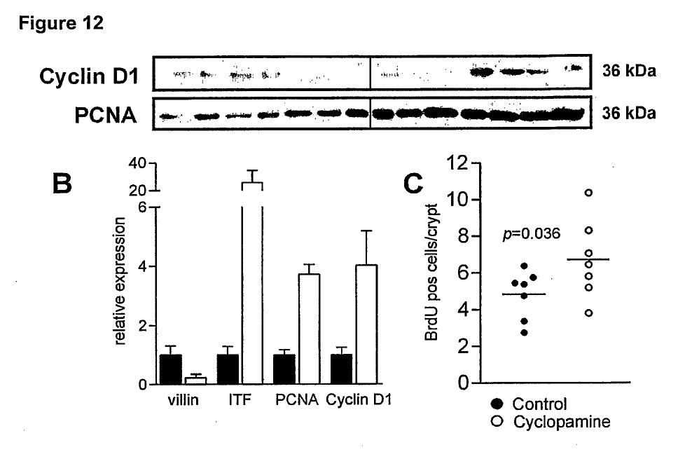

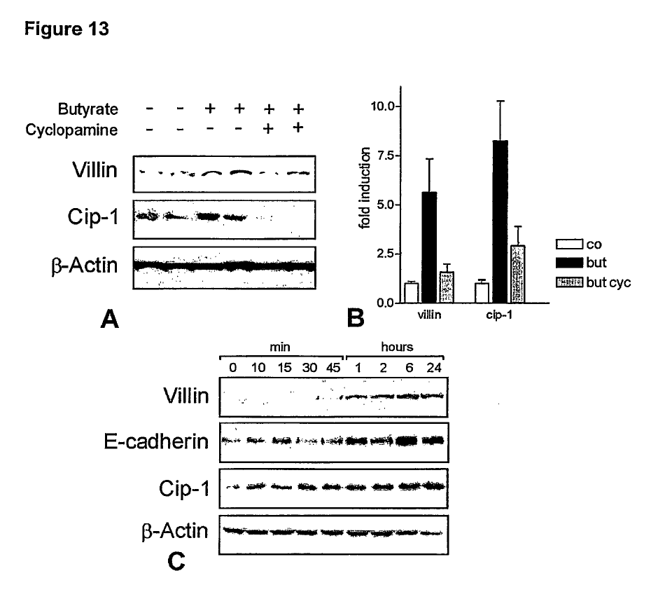

We first examined Shh and Ihh mRNA expression in the histological normal human colon (FIG. 1a) and both Shh and Ihh protein expression in humans, rats and mice (FIGS. 1b and c). In all three species the terminally differentiated absorptive enterocytes expressed Ihh. Although we found low levels of Shh mRNA at the bases of the colonic crypts, we were unable to demonstrate detectable Shh protein, whereas we readily detected Shh protein in both adult human stomach and zebrafish endoderm, used as positive control (not shown). To see if Ihh correlated with the differentiation state in an in vitro model of enterocyte differentiation, we next examined Ihh expression in butyrate-treated HT29 cells (Zweibaum et al., 1985). Induction of Ihh protein expression correlates well with expression of the differentiation marker Villin in this model (FIG. 1d).

To begin to understand the role of Hh signalling in the adult colon we focused on the vertebrate homologues of four Drosophila genes with an established role in hindgut formation. These are Dpp (vertebrate homologies BMP2 and BMP4), Fork Head (vertebrate homologue HNF3β/FoxA2), Serpent (vertebrate GATA factors) and Engrailed (vertebrate Engrailed-1 and 2). We localised the expression of these proteins by immunohistochemistry and determined their relation to the Hh signal in vivo in the rat using the Hh inhibitor cyclopamine. Cyclopamine is a potent Hh signalling inhibitor that inhibits Hh signalling at the level of Smo (Taipale et al., 2000). Since only Ihh protein is detectable in the adult colon, we presume that the effects of cyclopamine relate principally if not entirely to the inhibition of Ihh signalling.

We found that the Hh binding receptor and transcriptional target Ptc was broadly expressed in the epithelial cells along the crypt axis and on various mesenchymal cells (FIG. 2a). Thus Ihh may directly affect a wide range of target cells both within the epithelium and in the mesenchyme. Hh genes are often co-expressed with BMP2 and BMP4 (Bitgood et al., 1995; van den Brink et al., 2001) We found that the expression of both morphogens in the adult colon was similar to that found during embryonic and fetal development, BMP-2 is expressed by the differentiated enterocytes (FIG. 2b), whereas we detected BNP-4 in myofibroblast-like mesenchymal cells and in some epithelial cells in the proximal colon with endocrine cell morphology (FIGS. 2c and d). Whereas no effect was found on the expression levels of BMP-2 upon cyclopamine treatment, levels of BMP4 were markedly induced in response to Hh inhibition supporting the known interaction between the Hh and BMP signalling pathways.

The transcription factors HNF3β and En-1 are highly expressed in the epithelial cells at the base of the crypt, but this expression diminishes towards the Ihh expressing cells at the intercrypt tables (FIG. 2e). Both HNF3β and engrailed-1 are dramatically upregulated in response to cyclopamine treatment (FIG. 3). Of the GATA factors only GATA-6 has previously been found in colon cancer cells (Gao et al., 1998). In vivo, we observed GATA-6 expression in the terminally differentiated enterocytes at the intercrypt tables (not shown). Upon cyclopamine treatment GATA-6 was significantly downregulated (FIG. 3). Ihh signalling is not necessary for maintenance of its own expression (FIG. 3).

1.2. Cyclopamine Treatment Affects Both Differentiation and Proliferation In Vivo

We sought to determine if cyclopamine interfered with colonic epithelial cell differentiation. We studied the expression of markers of differentiation of the two main epithelial cell lineages of the colon, the enterocyte and the goblet cell. We observed a strong induction of Intestinal trefoil factor (ITF/TFF3), a goblet cell lineage marker (FIG. 4). In contrast, cyclopamine treatment reduced the expression of Villin, a cytoskeletal protein that is specific for microvilli and is a marker of enterocyte differentiation (Pringault et al., 1986). Inhibition of Hh signalling therefore seems to interfere with enterocyte differentiation in vivo, and promote the differentiation of the goblet cell lineage.

Finally we used three markers of proliferation to assess the effect of cyclopamine treatment on the precursor cell compartment. Cyclopamine treatment increased both the expression of the cyclin PCNA and cyclin D1 and the incorporation of 5-bromo-2′-deoxyuridine (BrdU, FIG. 4) showing that, in the adult colon, Hh signalling may negatively regulate precursor cell proliferation.

FIG. 11 further shows that cyclopamine treatment also disturbs enterocyte maturation in vivo. Treatment of rats with this inhibitor inhibited terminal differentiation of enterocytes in the colon, leading to a pre-malignant state as evident by dramatic changes in histoarchitecture, villin-redistribution, loss of carbonic anhydrase expression, and induction of expression of intestinal trefoil factor in the enterocytes. FIG. 12 shows that in accordance with the above described results on cell differentiation, cyclopamine treatment enhanced mitogenesis as judged from the expression of the cyclin PCNA and cyclin D1 and the number of 5-bromo-2′-deoxyuridine (BrdU) labelled epithelial precursor cells (FIG. 12c). We interpret these results as that Hh signalling negatively regulates precursor cell proliferation in the adult colon.

1.3. Loss of Ihh Expression is an Early Event in the Adenoma-Carcinoma Sequence

Since we found that Ihh plays a role in the differentiation of the enterocyte lineage, we were interested to examine Ihh expression in the adenoma to carcinoma sequence of colon carcinogenesis (FIG. 5). In resection specimens of histological normal tissue (n=3) we found Ihh staining of all the terminally differentiated enterocytes. In tubulovillous adenomatous polyps (n=8), Ihh staining was completely lost. In contrast, we found normal Ihh staining in the morphologically normal tissue present in 5 of these specimens. Of the 9 adenocarcinomas studied 6 were localised within an adenomatous polyp and 7 contained normal tissues. Ihh staining was detected in the normal epithelium but was lost in 8 out of 9 carcinomas and in all of the adenomatous areas. In one poorly differentiated mucinous adenocarcinoma, small clusters of poorly differentiated cells strongly reacted with the Ihh antibody. From these experiments it became evident that loss of Ihh expression already occurs at the polyp stage.

The very early lesions of the adenoma-carcinoma sequence were examined in 10 resection specimens of four different patients with FAP (familial adenomatous polyposis coli). These patients develop numerous adenomas due to a germline APC mutation, and somatic inactivation of the wild-type APC-allele. Multiple very small adenomas can be found in these specimens due to the frequent inactivation of both APC alleles in those patients.

We found that Ihh expression was clearly lost at the single crypt adenoma stage (not shown). In two different FAP patients we found areas with complete loss of Ihh staining in the epithelium of a few morphologically normal adjacent crypts (FIG. 5f). β-catenin staining of consecutive sections (FIG. 5g) demonstrated loss of membrane staining and cytoplasmic accumulation (FIG. 5i) of β-catenin in the same area. The aberrant localisation of β-catenin corresponds to that described previously in human aberrant crypt foci, the earliest morphologically recognisable putative premalignant lesion (Hao et al., 2001). We found that although there is no clear morphological change in these crypts yet, Wnt-β-catenin signalling is disturbed. Such crypts represent therefore the earliest (immunohistochemically) detectable stage following an APC mutation, loss of Ihh occurred at this earliest stage. Since this loss appears to precede microscopically detectable morphological change it may be an important step in the transition of the normal crypt into the adenoma. Furthermore, Ihh staining may be a useful tool to find these early lesions in large resection specimens since it identifies lesions that cannot be detected based on morphological criteria.

To study the very early lesions of the adenoma-carcinoma sequence, we examined 10 resection specimens of four different patients with FAP (familial adenomatous polyposis coli).

1.4. Recombinant hh Restores HT-29 Colonic Cancer Cell Differentiation In Vitro.

From our in vivo data it appeared that Ihh expression in the terminally differentiated enterocytes signals in an autocrine manner. Loss of Ihh expression was evident in the earliest recognisable stage in the sequence of events that is thought to progress to colon cancer. In vitro we find that Ihh is not expressed in malignant colonic epithelial cells. The colon cancer cell HT-29 will only express Ihh protein after differentiation with butyrate. We therefore decided to examine if HT-29 cells can be differentiated with recombinant Hh protein in vitro. We used recombinant Shh since this has a higher biological activity than recombinant Ihh, utilises the same receptor and induces the same biological response as Ihh (Pathi et al., 2001; Yang et al., 1998). Forty-eight hour treatment of HT-29 cells induces Villin expression to a similar extent as after treatment with 5 mM butyrate (FIG. 6). These data show that exogenous Hh protein is sufficient to restore differentiation of colon carcinoma cells.

1.5. Butyrate Induced Differentiation of HT-29 Cells is Ihh Dependent

Treatment of HT-29 cultures with 2 μ/ml cyclopamine significantly reduced butyrate mediated induction of cip-1, Villin and E-cadherin (FIGS. 13a,b). Also we were able to induce the same proteins (FIG. 13c) with 2.5 μg/ml recombinant N-terminal mature murine Shh peptide, which is 91% identical with the corresponding Ihh peptide, binds Ptc with the same affinity and induces the same biological response (Pathi et al., 2001). Thus Hh signalling is both necessary and sufficient for colon cancer cell differentiation. Together these results show that: (i) Ihh is expressed by the terminal enterocytes; (ii) that inhibiting Hh signalling in vivo impair enterocyte differentiation; (iii) loss of Ihh expression precedes the development of epithelial dysplasia; and (iv) that application of Hh to colon cancer cells restores differentiation. Thus these data demonstrate that Ihh is an important factor in the maintenance of colonic epithelial homeostasis and indicate an important role for Ihh in the earliest stages carcinogenesis in the colon.

### Example 2

### Gastric Tissues

2.1. Shh Expression is Fundic Gland Specific

We have previously shown that Shh protein is expressed in the fundic glands of humans and rodents (van den Brink et al., 2001). We have now examined the length of the human GI tract for both Shh mRNA and protein expression. While no Shh mRNA was found in the normal squamous epithelium of the adult human esophagus, Shh mRNA was abundantly expressed in the fundic part of the stomach and at low levels in the crypts of the small intestine and colon (as has been described in the human foetus, see ref 13) (FIG. 7). To investigate expression of Shh protein we have used an antibody that recognises the Shh precursor protein. In the human, mouse and rat GI tract Shh staining was exclusively detected in the fundic glands of the stomach. By contrast, no Shh staining was observed in the esophagus or the intestine (FIG. 8).

2.2. Shh Expression is Lost in Intestinal Metaplasia of the Fundus

During development of the stomach, absence of Shh leads to intestinal transformation of the stomach (de Santa Barbara et al.). In humans, replacement of gastric epithelium by epithelium of intestinal phenotype, or intestinal metaplasia, is commonly observed in patients with chronic gastritis. This metaplasia is an important risk factor for the development of gastric adenocarcinoma (Stemmermann, 1994). To evaluate the possibility that alterations in Shh expression may be involved in intestinal metaplasia in humans, we studied whether Shh expression is lost in intestinal metaplasia of the fundic gland region. To optimally localise Shh expression relative to the intestinal metaplasia in these specimens we used an immunohistochemical triple staining method. This method visualises cytoplasmic and extracellular MUC5AC, the mucin produced by gastric pit cells, cytoplasmic MUC2, a mucin specifically expressed by the goblet cells of the intestine (Tytgat et al., 1994), and Shh. This triple stain has an additional advantage that intestinal absorptive enterocytes are readily identified in specimens with intestinal metaplasia due to the typical thin staining of the brush border that contains intestinal alkaline phosphatase.

No overlap between MUC5AC and Shh was found since MUC5AC marks pit cells that migrate up from the precursor cell and Shh is exclusively expressed by the downward migrating parietal cells of the gastric glands (van den Brink et al., 2001). Using this method we found that the expression of Shh and MUC2 is mutually exclusive in all specimen investigated (n=16). This clearly demonstrates that in areas of intestinal metaplasia Shh expression is completely lost.

Although not the primary aim of our study, we were also able to identify three types of intestinal metaplasia on the basis of mucin expression, in the specimens that allowed good visualisation of the full gastric unit. The first type was characterised by MUC2 positive goblet cells and intestinal-type absorptive cells with an alkaline phosphatase positive brush border (see unit marked with asterisks in FIG. 9D). In the second type, only the gland cells were replaced by MUC2 expressing goblet cells whereas the pit cells where still of the MUC5AC expressing gastric phenotype (see FIGS. 9A-C). The third type consisted of cases the gland contained MUC2 positive goblet cells whereas the pit consisted of a mix of MUC5AC expressing pit cells and MUC2 positive goblet cells (see unit marked with arrow in FIG. 9D). Three types of goblet cells were observed in this study. Most goblet cells were found to express MUC2 exclusively (FIG. 9^{E}) as is the case in a normal intestinal goblet cell (Tytgat et al., 1994), however we also found more rarely that goblet cells can display exclusive expression of MUC5AC (FIG. 9G) or co-express MUC2 and MUC5AC (FIG. 9F). The MUC2-MUC5AC co-expressing goblet cells were found in the region of the isthmus, whereas goblet cells that had migrated further from here were invariably found to express only MUC2 indicating a transition from a mixed gastric-intestinal to a purely intestinal phenotype.

2.3. Shh is Expressed in Fundic Gland Heterotopia

To investigate if Shh is expressed in gastric heterotopia of the small intestine, we examined human resection specimens of Meckel's diverticulum (n=13). Meckel's diverticulum is a common abnormality of the small intestine that occurs in 1-3% of the population (Turgeon et al., 1990). This remnant of the omphalomesenteric duct often contains heterotopic tissue of various endodermal derivatives. We stained all specimens examined for both the H⁺K⁺ ATPase to identify acid producing parietal cells of the fundic gland and for Shh. All specimens that contained parietal cells (n=8) were also positive for Shh (FIGS. 10B,C), whereas specimens that lacked fundic glands (4 with intestinal and 1 with antral mucosa) also lacked Shh staining (FIG. 10A). Thus Shh is expressed in fundic gland heterotopia, indicating that aberrant development of intestinal epithelium into gastric epithelium with fundic glands is accompanied by Shh expression.

2.4. Shh is Expressed in Fundic Gland Metaplasia of the Esophagus

In patients with chronic acid reflux the resulting inflammation of the esophagus can lead to columnar metaplasia of the normally squamous epithelium of the esophagus, a condition called Barrett's esophagus. While these patients frequently develop intestinal metaplasia in the columnar lined segment, a mixture of gastric and intestinal-type epithelium is commonly observed (Jankowsk et al., 2000). To see if Shh expression is also induced postnatally in areas of gastric metaplasia we examined oesophageal resection specimens of 6 patients with Barrett's esophagus for expression of both the H⁺K⁺ ATPase and Shh. We found one resection specimen with areas of gastric metaplasia of fundic type glands. A complete overlap of H⁺K⁺ ATPase expression and Shh expression was found in this specimen (FIG. 10E), whereas all oesophageal (including the submucosal glands) and intestinal tissue in the resection specimens was negative for Shh (FIG. 10D). This indicates that the switch in differentiation from squamous to gastric epithelial tissue with fundic glands is accompanied by induction of Shh expression.

### Literature

Apelqvist A, Ahlgren U, Edlund H: Sonic hedgehog directs specialised mesoderm differentiation in the intestine and pancreas. Curr Biol. 1997;7:801-804.
Augeron, C. & Laboisse, C. L. Emergence of permanently differentiated cell clones in a human colonic cancer cell line in culture after treatment with sodium butyrate. Cancer Res. 44: 3961-3969 (1984).
Bedi, A. et al. Inhibition of apoptosis during development of colorectal cancer. Cancer Res. 55, 1811-1816 (1995).
Bitgood, M. J. & MacMahon, A. P. Hedgehog and Bmp genes are coexpressed at many diverse sites of cell-cell interaction in the mouse embryo. Dev. Biol. 172, 126-138 (1995).
Bossard P, Zaret K S: Repressive and restrictive mesodermal interactions with gut endoderm: possible relation to Meckel's Diverticulum. Development 2000;127:4915-23.
Burdick J S, Chung E, Tanner G, Sun M, Paciga J E, Cheng J Q, Washington K, Goldenring J R, Coffey R J: Treatnent of Menetrier's disease with a monoclonal antibody against the epidermal growth factor receptor. N Engl J Med. 2000;343:1697-1701
Chang, W. W. & Leblond, C. P. Renewal of the epithelium in the descending colon of the mouse. I. Presence of three cell populations: vacuolated-columnar, mucous and argentaffin. Am. J. Anat. 131, 73-99 (1971).
D'Elios M M, Bergman M P, Azzurri A, Amedei A, Benagiano M, De Pont J J, Cianchi F, Vandenbroucke-Grauls C M, Romagnani S, Appelmelk B J, Del Prete G: H(+),K(+)-atpase (proton pump) is the target autoantigen of Th1-type cytotoxic T cells in autoimmune gastritis. Gastroenterology. 2001;120:377-386
Dempsey P J, Goldenring J R, Soroka C J, Modlin I M, McClure R W, Lind C D, Ahlquist D A, Pittelkow M R, Lee D C, Sandgren E P, Page D P, Coffey R J: Possible role of transforming growth factor alpha in the pathogenesis of Menetrier's disease: supportive evidence form humans and transgenic mice. Gastroenterology. 1992;103:1950-1963.
Fan, H. & Khavari, P. A. Sonic hedgehog opposes epithelial cell cycle arrest. J. Cell. Biol. 147, 71-76 (1999).
Finn L S, Christie D L: Helicobacter pylori and Meckel's diverticula. J Pediatr Gastroenterol Nutr 2001;32:150-155
Fodde, R. et al. Mutations in the APC tumour suppressor gene cause chromosomal instability. Nat. Cell. Biol. 3, 433-438 (2001).
Gao, X. Sedgwick, T. Shi, Y. B. & Evans, T. Distinct functions are implicated for the GATA4, -5, and -6 transcription factors in the regulation of intestine epithelial cell differentiation. Mol. Cell. Biol. 18, 2901-2911 (1998).
Gordon J I, Hermiston M L: Differentiation and self-renewal in the mouse gastrointestinal epithelium. Curr Biol 1994;6:795-803
Hall, P. A. Coates, P. J. Ansari, B. & Hopwood, D. Regulation of cell number in the mammalian gastrointestinal tract: the importance of apoptosis. J. Cell. Sci. 107, 3569-3577 (1994).
Hao, X. P. Pretlow, T. G. Rao, J. S. & Pretlow, T. P. beta-Catenin Expression Is Altered in Human Colonic Aberrant Crypt Foci. Cancer Res. 61, 8085-8088 (2001).
Hebrok M, Kim S K, Melton D A: Notochord repression of endodernal Sonic hedgehog permits pancreas development. Genes Dev. 1998;12:1705-1713
Hermiston, M. L., Wong, M. H., & Gordon, J. I. Forced expression of E-cadherin in the mouse intestinal epithelium slows cell migration and provides evidence for nonautonomous regulation of cell fate in a self-renewing system. Genes Dev. 10, 985-996 (1996).
Hoch, M. & Pankratz, M. J. Control of gut development by fork head and cell signalling molecules in Drosophila. Mech. Dev. 58, 3-14 (1996).
Hogan, B. L. Morphogenesis. Cell 96,225-233 (1999).
Incardona, J. P. Gaffield, W. Kapur, R. P. & Roelink, H. The teratogenic Veratrum alkaloid cyclopamine inhibits Sonic hedgehog signal transduction. Development 125, 3553-3562 (1998).
Jankowski J A, Harrison R F, Perry I, Balkwill F, Tselepis C: Barrett's metaplasia. Lancet 2000;356:2079-2085.
Kalderon D: Transducing the hedgehog signal. Cell. 2000;103:371-374
Karam S M, Leblond C P: Identifying and counting epithelial cell types in the “corpus” of the mouse stomach. Anat. Rec. 1992;232:231-246
Karam S M, Li Q, Gordon J I: Gastric epithelial morphogenesis in normal and transgenic mice. Am J Physiol. 1997;272:G1209-1220
Kenney, A. M. & Rowitch, D. H. Sonic hedgehog promotes G(1) cyclin expression and sustained cell cycle progression in mammalian neuronal precursors. Mol. Cell. Biol. 20, 9055-9067 (2000).
Kim S K, Melton D A: Pancreas development is promoted by cyclopamine, a hedgehog signalling inhibitor. Proc Natl Acad Sci USA. 1998;27:13036-13041
Kinzier, K. W. & Vogelstein, B. Lessons from hereditary colorectal cancer. Cell 87, 159-170 (1996).
Komorowski R A, Caya J G: Hyperplastic gastropathy. Clinicopathologic correlation. Am J Surg Pathol 1991;15:577-585
Levin M, Johnson R L, Stern C D, Kuehn M, Tabin C: A molecular pathway determining left-right asymmetry in chick embryogenesis. Cell. 1995;82:803-814.
Li Q, Karam S M, Coerver K A, Matzuk M M, Gordon J I: Stimulation of activin receptor II signalling pathways inhibits differentiation of multiple gastric epithelial lineages. Mol Endocrinol 1998;12:181-192
Li Q, Karam S M, Gordon J I: Diphtheria toxin-mediated ablation of parietal cells in the stomach of transgenic mice. J Biol Chem 1996;271 :3671-3676
Litingung Y, Lei L, Westphal H, Chiang C: Sonic hedgehog is essential to foregut development. Nat. Genet. 1998;20:58-61
Marigo, V. et al. Cloning, expression, and chromosomal location of SHH and IHH: two human homologues of the Drosophila segment polarity gene hedgehog. Genoinics 28, 44-51 (1995). Hogan BLM: Morphogenesis. Cell 1999;96:225-233
McMahon A P. More surprises in the Hedgehog signalling pathway. Cell 100, 185-188 (2000).
Montgomery, R. K. Mulberg, A. E. & Grand, R. J. Development of the human gastrointestinal tract: twenty years of progress. Gastroenterology 116, 702-731 (1999).
Moser, A. R. Dove, W. F. Roth, K. A. & Gordon, J. I. The Min (multiple intestinal neoplasia) mutation: its effect on gut epithelial cell differentiation and interaction with a modifier system. J. Cell Biol. 116, 1517-1526 (1992).
Nuisslein-Volhard C, Wieschaus E: Mutations affecting segment number and polarity in Drosophila. Nature 1980;287:795-801
Oshima, H. Oshima, M. Kobayashi, M. Tsutsumi, M. & Taketo, M. M. Morphological and molecular processes of polyp formation in Apc(delta716) knockout mice. Cancer Res. 57, 1644-1649 (1997).
P. de Santa Barbara, G. R. van den Brink, & D. J. Roberts. The molecular etiology of gut malformations and diseases. Semin. Med. Genet. in press.
Pathi, S. et al. (2001). Comparative biological responses to human Sonic, Indian, and Desert hedgehog. Mech. Dev. 106, 107-117.
Pringault, E. M. Arpin, M. Garcia, A. Findori, A. & Louvard, D. A. Human villin cDNA clone to investigate the differentiation of intestinal and kidney cells in vivo. EMBO J. 5:3119-3124 (1986).
Rainalho-Santos, M. Melton, D. A. McMahon, A. P. Hedgehog signals regulate multiple aspects of gastrointestinal development. Development 127, 2763-2772 (2000).
Reis C A, David L, Correa P, Carneiro F, de Bolos C, Garcia E, Mandel U, Clausen H, Sobrinho-Simoes M: Intestinal metaplasia of human stomach displays distinct patterns of mucin (MUC1, MUC2, MUC5AC, and MUC6) expression. Cancer Res. 1999;59: 1003-1007
Risio, M. et al. Correlations between rectal mucosa cell proliferation and the clinical and pathological features of nonfamilial neoplasia of the large intestine. Cancer Res. 51, 1917-1921 (1991).
Roberts D J, Johnson R L, Burke A C, Nelson C E, Morgan B A, Tabin C J: Sonic hedgehog is, an endodermal signal inducing BMP-4 and Hox genes during induction and regionalization of the chick hindgut. Development 1995; 118:47-59
Roberts, D. J. et al. Sonic hedgehog is an endodermal signal inducing Bmp-4 and Hox genes during induction and regionalization of the chick hindgut. Development 121, 3163-3174 (1995).
Roberts, D. J. Molecular mechanisms of development of the gastrointestinal tract. Dev Dyn. 219, 109-120 (2000).
Roberts, D. J. Smith, D. M. Goff, D. J. & Tabin, C. J. Epithelial-mesenchymal signalling during the regionalization of the chick gut. Development 125, 2791-2801 (1998).
Ryan A K, Blumberg B, Rodriguez-Esteban C, Yonei-Tamura S, Tamura K, Tsukui T, de la Pena J, Sabbagh W, Greenwald J, Choe S, Norris D P, Robertson E J, Evans R M, Rosenfeld M G, Izpisua Belmonte J C: Pitx2 determines left-right asymmetry of internal organs in vertebrates. Nature. 1998;394:545-551.

1. A method of treating a deficiency of a Hedgehog protein in the GI tract of a subject deficient in said protein and in need of such treatment, comprising providing to the GI tract of the subject a composition that comprises a source of a Hedgehog protein.

2. A method according to claim 30, wherein the subject is at risk for, or suffers from cancer of the GI tract and said providing results in
(i) prevention of said cancer development, or
(ii) therapy of said cancer.

3. A method according to claim 2, wherein the cancer is is gastric or colon cancer.

4. A method according to claim 2, wherein the from a cancer is a GI tract carcinoma and said providing results in therapy of said carcinoma.

5. A method according to claim 4, wherein the GI tract carcinoma is a gastric or colon carcinoma.

6. A method according to claim 1, wherein the deficiency of the Hedgehog protein is an acquired deficiency.

7. A method according to claim 30, wherein the subject has familial adenomatous polyposis coli (FAP).

8. A method according to claim 7, wherein the Hedgehog protein, homologue or variant, nucleic acid expression vector, bacterial delivery vehicle, animal cell or inducing or upregulating agent, is administered in an amount effective to prevent or reverse GI tract tumorigenesis in the FAP subject.

9. A method according to claim 7, wherein the Hedgehog protein, homologue or variant, nucleic acid expression vector, bacterial delivery vehicle, animal cell or inducing or upregulating agent, is administered in an amount effective to prevent or reverse colonic adenomatous polyps, invasive adenocarcinomas, small intestinal adenomas and cancers, and desmoid tumors.

10. A method according to claim 30, wherein the source of Hedgehog protein comprises the Hedgehog protein, homologue or variant in a pharmaceutical composition.

11. A method according to claim 10, wherein the pharmaceutical composition is suitable for oral administration.

12. A method according to claim 30, wherein the source of the Hedgehog protein comprises said nucleic acid vector in a pharmaceutical composition.

13. A method according to claim 30, wherein the source of Hedgehog protein comprises said enteric bacterium.

14-24. (canceled)

25. A method for determining whether subject is at risk for developing a GI tract tumor comprising measuring the level of a Hedgehog protein or a Hedgehog mRNA in a GI tract tissue sample from the subject.

26. A method according to claim 25, wherein the tissue sample is a gastric, esophageal or colon tissue.

27. A method for diagnosing (i) the susceptibility of a subject to develop, or (ii) the presence in a subject ofi ectopic gastric tissue comprising, determining the level of Hedgehog, BMP2 or BMP4 mRNA and/or protein in a tissue sample from the subject.

28. A therapeutic composition comprising a nucleotide sequence encoding a Hedgehog protein which composition is in the form of.
(a) a nucleic acid expression vector; or
(b) an enteric bacterium comprising the nucleotide sequence which bacterium is capable of secreting the Hedgehog protein when colonizing the GI system of a subject.

29. The therapeutic composition of claim 28 which is said enteric bacterium .

30. The method of claim 1 wherein said source of Hedgehog protein is:
(a) a Hedgehog protein or an active homologue or variant thereof;
(b) a nucleic acid expression vector comprising a nucleotide sequence that encodes said protein, homologue or variant;
(c) an enteric bacterium capable of colonizing a part of the GI tract which is transformed with said encoding nucleotide sequence and expresses and secretes said Hedgehog protein, homologue or variant;
(d) an animal cell that expresses and secretes said Hedgehog protein, homologue or variant; or
(e) a molecule or agent that induces or upregulates expression of Hedgehog protein in said subject.

31. The method of claim 30 wherein said Hedgehog protein is Shh or Ihh and said homologue or variant is a homologue or variant of Shh or Ihh.

32. A method for preventing the development or treating a disease or condition characterized by the presence or growth of a Hedgehog protein-expressing ectopic gastric tissue in a subject, comprising providing to the subject or to the site of said ectopic tissue an effective amount of a substance that reduces the functional level or activity of the Hedgehog protein.

33. A method according to claim 32, wherein said substance is an antibody specific for said Hedgehog protein or an antisense nucleic acid at least part of which is complementary to at least a functional part of a Hedgehog mRNA that encodes a Hedgehog protein selected from the group consisting of human Desert Hedgehog protein SEQ ID NO:1, human Indian Hedgehog protein SEQ ID NO:2, and human Sonic Hedgehog protein SEQ ID NO:3.


SEQUENCE LISTING (3 sequences)

SEQ ID NO: 1 - protein, 396 aa, Homo sapiens
  Human Desert Hedgehog protein
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 2 - protein, 293 aa, Homo sapiens
  Human Indian Hedgehog protein
MNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 3 - protein, 462 aa, Homo sapiens
  Human Sonic hedgehog protein
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS